(19) United States
(12) Patent Application Publication
(10) Pub. No.: US 2010/0099917 A1
(43) Pub. Date: Apr. 22, 2010

(54) Integrin modulators and methods for their use

(75) Inventors: John B. Davidson, Chicago, IL (US); Ali S. Turgiev, Moscow (RU)

Correspondence Address: BRINKS HOFER GILSON & LIONE, P.O. BOX 10395, CHICAGO, IL, 60610, US

(73) Assignee: Billings Pharmaceuticals, Inc.

(21) Appl. No.: 12/455,742

(22) Filed: Jun. 5, 2009

Related U.S. Application Data
(63) Continuation of application No. 10/592,212, filed on Sep. 8, 2006, now abandoned.
(60) Provisional application No. 60/552,239, filed on Mar. 10, 2004.

(51) Int. Cl. C07C 317/08

(52) U.S. Cl. 568/36

(57) ABSTRACT

1,5-Dithiaocta-2,7-diene-5-oxide-1-yl(DODOyl) compounds and derivatives thereof, referred to collectively as DODOyl-derived compounds (DDCs), and chiral enantiomers and derivatives thereof, are described, which are integrin modulators that modulate integrin-mediated functions and/or processes. Pharmaceutical compositions containing integrin modulators and chiral enantiomers thereof, and methods for using integrin modulators and chiral enantiomers thereof are further described.

### FIELD OF THE INVENTION

The present invention relates to agents for modulating one or more integrin-mediated functions, and to methods for their use.

### BACKGROUND

Integrins are heterodimeric transmembrane glycoproteins which, inter alia, act as cell receptors for various entities, herein termed collectively “integrin ligands,” including, for example, surface molecules of other cells and extracellular matrix (ECM) proteins. Both soluble and immobilized integrin ligands are known to be ordinarily bound by integrins. Integrins are found on most types of cells. Various cellular events in which integrins may participate or which are associated with integrins or integrin activity are referred to collectively herein as “integrin-mediated” events. For a general review of integrins, see: Guidebook to the Extracellular Matrix and Adhesion Proteins (Kreis et al., Eds., 1993), and The Adhesion Molecule Facts Book (Pigot et al., Academic Press, 1993).

One such integrin-mediated cellular function is signaling. For instance, certain integrins are known to participate in the transfer of information from the inside to the outside of the cell (inside-out signaling) or from the outside to the inside of the cell (outside-in signaling), although other types of signaling may also occur, as may combinations thereof. An example involving inside-out signaling is the process whereby an integrin acquires or expresses affinity for ligands in response to intracellular events (integrin upregulation). Binding of integrin ligands to certain integrins (e.g., in the case of integrin-mediated cell adhesion) may initiate signal transduction events, in a manner similar to that described for other cell surface receptors. Signals thus elicited are termed outside-in signals and are involved in the regulation of various cell responses, which may include gene expression, cell differentiation, and cell proliferation.

Signaling may result in the clustering of cellular molecules in localized areas of cellular membrane, for example, in the association of integrins with each other (and other molecules) by interactions. The formation of such clusters may influence various integrin-mediated functions in multiple ways, including, for example, by additional or secondary signaling events or interactions, and by altered ligand affinity.

The integrin-mediated function of adhesion is, or various integrin-mediated events associated with adhesion are, important for a variety of physiological and pathological responses. The extent of adhesion is functionally related to integrin-mediated signaling. For example, in association with initial integrin-dependent adhesion to a substratum, certain cells change their shape and start spreading on the surface of the stratum, employing integrins in the process of establishing new contacts with the underlying proteins (e.g., extracellular matrix [ECM] components). In motile cells, the whole array of integrin-mediated events involving adhesion—initial contact, cell shape change, cell spreading, and cell locomotion—is sometimes termed “the adhesion cascade” (S. R. Sharar et al., “The Adhesion Cascade and Anti-Adhesion Therapy: An Overview,” Springer Semin. Immunopathol. 1995, 16, 359). Adhesion cascades are viewed as integral to one or more familiar cell motility patterns, including angiogenesis, lymphocyte homing, tumor cell metastasis, and cell migration processes associated with wound healing, although similar cascade mechanisms are also viewed as operative even in the absence of cell locomotion (e.g., in platelet adhesion and aggregation). Extravasation (diapedesis) of neutrophils is described below in greater detail, as a paradigmatic integrin-mediated adhesion cascade (E. Hub et al., “Mechanism of Chemokine-Induced Leukocyte Adhesion and Emigration,” Chemoattractant Ligands and Their Receptors (R. Horuk, Ed., CRC Press, Boca Raton, 1996, 301).

The onset of extravasation is heralded by the appearance in the circulation of chemotactic factors, or chemoattractants (i.e., specific substances that initiate cell migration along their concentration gradients). Chemoattractants (e.g., chemokines, bacterial peptides, and products of complement activation) activate neutrophils to upregulate their integrin receptors (neutrophil integrins include, for example, LFA-1 [CD11a/CD18], CR3 [also known as Mac-1, CD11b/CD18], and gp154,95 [CD11c/CD18]). Neutrophils thus activated adhere to endotheliocytes, change shape, and spread on the endothelial surface. Thereafter, the stimulated motile apparatus of the neutrophils gives rise to migration, and the neutrophils start moving, first across the endothelial layer and further, through the perivascular ECM, towards the source of the chemotactic stimulus (e.g., pathogenic bacteria invading a certain bodily tissue). During the whole process, from the initial firm contact with the endothelium to the cessation of locomotion at the destination site, various integrins participate in the attachment of the neutrophil to the substrata it encounters, enabling its recruitment to the locus of infection. Parallel processes utilizing integrins are involved when cancer cells migrate to initiate metathetic foci in the body.

Another integrin-mediated function is cell-cell fusion. Under physiological conditions, fusion is a developmentally regulated stage in the differentiation of certain multinucleate cells (e.g., osteoclasts, myocytes, and syncytiotrophoblasts), and fusion is also a prerequisite to fertilization (in the case of sperm-egg fusion). Fusion is effected by specialized cellular systems involving integrins (e.g., refs. cited in: A.-P. J. Huovila, et al., “ADAMs and Cell Fusion,” Current Opin. Cell. Biol. 1996, 8, 692 and S. Ohgimoto et al., “Molecular Characterization of Fusion Regulatory Protein-1 [FRP-1] that Induces Multinucleate Giant Cell Formation of Monocytes and HIV gp160-Mediated Cell Fusion: FRP-1 and 4F2/CD98 Are Identical Molecules,” J. Immunol. 1995, 155, 3585).

The ability to undergo recirculation from intracellular compartments to the cell surface and vice versa is a common property of diverse cellular receptors, including integrins and integrin components (e.g., see: P. Handagama et al., “Kistrin, an Integrin Antagonist, Blocks Endocytosis of Fibrinogen into Guinea-Pig Megakaryocyte and Platelet alpha-Granules,” J. Clin. Invest. 1993, 91, 193). This capability facilitates the mediation of other cellular functions by transporting into the cell extracellular material (e.g., soluble proteins, particulate matter, and other cells). Integrin-mediated internalization is used by certain microorganisms to invade their targets. For example, CR3 mediates entry of iC3b-opsonized HIV-1 and HIV-2 into CD4-negative lymphocytic and monocytic cells (V. Boyer et al., “Complement Mediates Human Immunodeficiency Virus Type 1 Infection of a Human T cell Line in a CD4- and Antibody-Independent Fashion,” J. Exp. Med. 1991, 173, 1151).

The above-delineated integrin-mediated functions are illustrative only, as other characterizations of integrin-mediated functions can also be made. Moreover, the integrin-mediated functions as delineated herein are overlapping and interrelated. In the case of neutrophil extravasation, for example, the initial chemotactic signal activating the cells is commonly functionally associated with integrin upregulation (inside-out signaling) and adhesion to the endothelial surface. This adhesion event, in turn, is associated with an outside-in signal, enabling the neutrophil to change its shape, which is a prerequisite to the spreading and migration of the cell. Likewise, when the neutrophil that has arrived to the source of chemoattractants establishes an adhesive interaction with the bacteria by means of integrins, an outside-in signal is transduced, which is associated with the initiation of internalization of the integrins involved, together with the bacteria attached thereto (phagocytosis).

Furthermore, regarding outside-in integrin-mediated signaling, certain cellular processes are co-mediated by several signaling systems acting in concert. In the case of neutrophils extravasating to the tissues to phagocytose bacteria, the neutrophils receive signals by means of the receptors of the chemoattractant (along the concentration gradient of which the movement occurs) and by means of distinct integrins (including those that attach it to the substratum and, subsequently, those recognizing the bacteria). This interplay of signals mediates the antibacterial machinery of the neutrophils with the consequence that only upon contact with the bacteria, which is mediated by means of a particular type of integrin, are the constituents of the intracellular granules released and reactive oxygen species formed. As a result, the formation and release of microbicidal substances take place preferentially at sites of contact with bacteria, enabling effective killing of the bacteria and preventing the destruction of host tissue (S. D. Wright, “Receptors for Complement and the Biology of Phagocytosis” in Inflammation: Basic Principles and Clinical Correlates, 2^{nd} Ed. (J. I. Gallin et al., Eds., Raven Press, New York, 1992, Chapter 25, 477).

A broad range of cellular activities can be regulated by modulating certain integrin-mediated functions with appropriate agents. One such modulating agent is ajoene (4,5,9-trithiadodeca-1,6,11-triene-9-oxide):

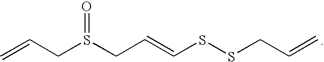

Ajoene, and a precursor thereof, can be formed via reactions involving products derived from garlic (Allium sativum). As an example, when garlic is crushed, cellular alliin may come into contact with alliinase in the cell wall to form allicin. In the presence of an appropriate polar solvent, allicin may then form ajoene. Ajoene has been previously shown to inhibit platelet aggregation by allosterically inactivating the platelet integrin, GP IIb/IIIa (R. Apitz-Castro et al., Biophys. Res. Commun. 1986, 141, 145). It has been demonstrated that stereoisomers at the internal double bond of ajoene (i.e., E- and Z-4,5,9-trithiadodeca-1,6,11-triene-9-oxides) exhibit no significant differences in their effects on platelets (E. Block et al., J. Am. Chem. Soc. 1986, 108, 7045). For this reason, most of the subsequent studies of the integrin modulation by ajoene were carried out on mixtures (typically designated as racemic) of the E- and Z-isomers. It was shown, for example, that ajoene is a potent inhibitor of a wide variety of adhesion-dependent processes, including neutrophil aggregation, HIV transmission (A. V. Tatarintsev et al., AIDS 1992, 6, 1215), and tumor metastasis. United States patents issued to Tatarintsev et al. describe the use of ajoene for treatment of inflammation (U.S. Pat. No. 5,948,821), arthritis (U.S. Pat. No. 5,856,363), and tumors (U.S. Pat. No. 5,932,621), as well as for contraception (U.S. Pat. No. 5,863,954) and inhibition of immune responses (U.S. Pat. No. 5,863,955). All of these diseases and conditions are believed to involve integrin-mediated processes. Ajoene has also been used to treat additional diseases and conditions which are believed to involve integrin-mediated processes, as described in PCT application WO 97/25031.

The presence in ajoene of the 6-double bond generates, depending on its geometry, two stereoisomers, E- and Z-ajoenes. The presence of the chiral 9-sulfoxide group generates the possibility for two optical isomers (enantiomers) of each stereoisomer. Thus, the above structural features of ajoene create the possibility for four enantiomers: (6E,9R), (6E,9S), (6Z,9R), and (6Z,9S)-4,5,9-trithiadodeca-1,6,11-triene-9-oxides. However, in the case of allicin (which also contains a sulfoxide functionality), the existence of optical activity has been questioned, so that the existence of enantiomers, or at least stable enantiomers, would have been considered unlikely (L. D. Lawson et al., Eds., “Garlic: The Science and Therapeutic Application of Allium Sativum L. and Related Species,” 1997, page 56).

### SUMMARY

The scope of the present invention is defined solely by the appended claims, and is not affected to any degree by the statements within this summary.

By way of introduction, a first compound embodying features of the present invention has a structure:

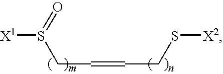

wherein:

X¹ and X² are the same or different and are each independently selected from the group consisting of hydrogen, an alkyl, an alkenyl, an alkynyl, an aryl, an aryl substituted with one or more —NO₂ groups, an aryl substituted with one or more lower alkyls, a group of formula RO—, a group of formula RC(O)—, a group of formula RC(O)O—, a group of formula ROC(O)—, a group of formula (R)₂N—, a group of formula RC(O)N—, a group of formula R(NH)C(O)—, a group of formula RN═N—, a group of formula RSO₂—, a group of formula RS(O)—, RSC(O)—, a group of formula RSO2O—, a group of formula RS(O)O—, a halogen atom, a nitroso group, a furanose unit, a pyranose unit, a combination of furanose units, a combination of pyranose units, a combination of furanose and pyranose units, and combinations thereof;

R is independently in each occurrence selected from the group consisting of a hydrogen, a lower alkyl, a lower alkenyl, an aryl, an aryl substituted with one or more lower alkyls, S-cysteinyl, peptidyl, an alkylphosphoglyceryl, an alkenylphosphoglyceryl, or an acylphosphoglyceryl;

m is an integer from 0 to 30; and n is an integer from 0 to 30;

providing that when X¹ is CH₂═CH—CH₂—, m is 1, and n is 0, X² is not CH₂═CH—CH₂—S— or (NH₂)—CH(CO₂H)—CH₂—S—.

A second compound embodying features of the present invention has a structure:

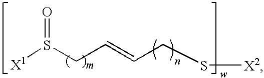

wherein:

X¹ is selected from the group consisting of hydrogen, an alkyl, an alkenyl, an alkynyl, an aryl, an aryl substituted with one or more —NO₂ groups, an aryl substituted with one or more lower alkyls, a group of formula RO—, a group of formula RC(O)—, a group of formula RC(O)O—, a group of formula ROC(O)—, a group of formula (R)₂N—, a group of formula RC(O)N—, a group of formula R(NH)C(O)—, a group of formula RN═N—, a group of formula RS—, a group of formula RSO₂—, a group of formula RS(O)—, RSC(O)—, a group of formula RSO2O—, a group of formula RS(O)O—, a halogen atom, a nitroso group, a furanose unit, a pyranose unit, a combination of furanose units, a combination of pyranose units, a combination of furanose and pyranose units, and combinations thereof;

X² is a polymeric species comprising a plurality of binding sites;

R is independently in each occurrence selected from the group consisting of a hydrogen, a lower alkyl, a lower alkenyl, an aryl, an aryl substituted with one or more lower alkyls, S-cysteinyl, peptidyl, an alkylphosphoglyceryt, an alkenylphosphoglyceryl, or an acylphosphoglyceryl; and

m is an integer from 0 to 30; n is an integer from 0 to 30; and w is an integer from 2 to 1000.

A third compound embodying features of the present invention has a structure:

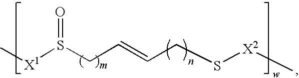

wherein X¹ and X² are the same or different and comprise at least one functional group configured for participation in a polymerization reaction;

wherein m is an integer from 0 to 30;

wherein n is an integer from 0 to 30; and

wherein w is an integer from 2 to 1000.

A fourth compound embodying features of the present invention has a structure:

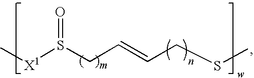

wherein X¹ comprises at least one functional group configured for participation in a polymerization reaction;

wherein m is an integer from 0 to 30;

wherein n is an integer from 0 to 30; and

wherein w is an integer from 2 to 1000.

A fifth compound embodying features of the present invention has a structure:

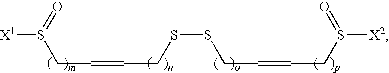

wherein X¹ and X² are the same or different and are each independently selected from the group consisting of hydrogen, an alkyl, an alkenyl, an alkynyl, an aryl, an aryl substituted with one or more —NO₂ groups, an aryl substituted with one or more lower alkyls, a group of formula RO—, a group of formula RC(O)—, a group of formula RC(O)O—, a group of formula ROC(O)—, a group of formula (R)₂N—, a group of formula RC(O)N—, a group of formula R(NH)C(O)—, a group of formula RN═N—, a group of formula RSO₂—, a group of formula RS(O)—, RSC(O)—, a group of formula RSO₂O—, a group of formula RS(O)O—, a halogen atom, a nitroso group, a furanose unit, a pyranose unit, a combination of furanose units, a combination of pyranose units, a combination of furanose and pyranose units, and combinations thereof;

R is independently in each occurrence selected from the group consisting of a hydrogen, a lower alkyl, a lower alkenyl, an aryl, an aryl substituted with one or more lower alkyls, S-cysteinyl, peptidyl, an alkylphosphoglyceryl, an alkenylphosphoglyceryl, or an acylphosphoglyceryl;

m is an integer from 0 to 30;

n is an integer from 0 to 30;

o is an integer from 0 to 30; and

p is an integer from 0 to 30.

A sixth compound embodying features of the present invention has a structure:

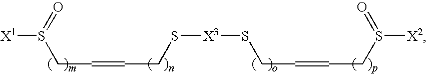

wherein X¹ and X² are the same or different and are each independently selected from the group consisting of hydrogen, an alkyl, an alkenyl, an alkynyl, an aryl, an aryl substituted with one or more —NO₂ groups, an aryl substituted with one or more lower alkyls, a group of formula RO—, a group of formula RC(O)—, a group of formula RC(O)O—, a group of formula ROC(O)—, a group of formula (R)₂N—, a group of formula RC(O)N—, a group of formula R(NH)C(O)—, a group of formula RN═N—, a group of formula RSO₂—, a group of formula RS(O)—, RSC(O)—, a group of formula RSO₂O—, a group of formula RS(O)O—, a halogen atom, a nitroso group, a furanose unit, a pyranose unit, a combination of furanose units, a combination of pyranose units, a combination of furanose and pyranose units, and combinations thereof;

R is independently in each occurrence selected from the group consisting of a hydrogen, a lower alkyl, a lower alkenyl, an aryl, an aryl substituted with one or more lower alkyls, S-cysteinyl, peptidyl, an alkylphosphoglyceryl, an alkenylphosphoglyceryl, or an acylphosphoglyceryl;

X³ is selected from the group consisting of an alkylene group and a metal atom;

m is an integer from 0 to 30;

n is an integer from 0 to 30;

o is an integer from 0 to 30; and

p is an integer from 0 to 30.

A pharmaceutical composition embodying features of the present invention includes a pharmaceutically-acceptable carrier and at least one of the compounds described above.

A method of modulating an integrin-mediated function of one or more cells that embodies features of the present invention includes contacting at least one of the cells with at least one of the compounds described above in an amount effective to modulate the integrin-mediated function.

A method of treating or preventing a disorder, disease or condition involving an integrin-mediated function that embodies features of the present invention includes administering to an animal in need thereof an effective amount of at least one of the compounds described above.

A method of treating or preventing at least one of a thrombotic disorder, disease or condition arising therefrom that embodies features of the present invention includes administering to an animal in need thereof an effective amount of at least one of the compounds described above.

A method of treating or preventing inflammation or an inflammatory disease that embodies features of the present invention includes administering to an animal in need thereof an effective amount of at least one of the compounds described above.

A method of treating, preventing, or inhibiting transmission of a viral infection that embodies features of the present invention includes administering to an animal in need thereof an effective amount of at least one of the compounds described above.

A method of treating psoriasis that embodies features of the present invention includes administering to an animal in need thereof an effective amount of at least one of the compounds described above.

A method of treating atherosclerosis that embodies features of the present invention includes administering to an animal in need thereof an effective amount of at least one of the compounds described above.

A method of treating cancer, preventing metastasis of tumors, or inhibiting integrin-mediated carcinogenesis that embodies features of the present invention includes administering to an animal in need thereof an effective amount of at least one of the compounds described above.

An integrin-modulator embodying features of the present invention includes a compound that forms at least one moiety in vivo, wherein the moiety has a structure:

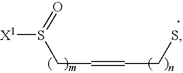

wherein:

X¹ is selected from the group consisting of hydrogen, an alkyl, an alkenyl, an alkynyl, an aryl, an aryl substituted with one or more —NO₂ groups, an aryl substituted with one or more lower alkyls, a group of formula RO—, a group of formula RC(O)—, a group of formula RC(O)O—, a group of formula ROC(O)—, a group of formula (R)₂N—, a group of formula RC(O)N—, a group of formula R(NH)C(O)—, a group of formula RN═N—, a group of formula RS—, a group of formula RSO₂—, a group of formula RS(O)—, RSC(O)—, a group of formula RSO2O—, a group of formula RS(O)O—, a halogen atom, a nitroso group, a furanose unit, a pyranose unit, a combination of furanose units, a combination of pyranose units, a combination of furanose and pyranose units, and combinations thereof;

R is selected from the group consisting of a hydrogen, a lower alkyl, a lower alkenyl, an aryl, an aryl substituted with one or more lower alkyls, S-cysteinyl, peptidyl, an alkylphosphoglyceryl, an alkenylphosphoglyceryl, or an acylphosphoglyceryl;

m is an integer from 0 to 30; and

n is an integer from 0 to 30;

providing that the compound is not ajoene or ajocysteine.

A liposome preparation embodying features of the present invention includes a compound having a formula:

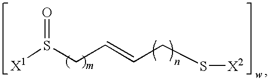

wherein X¹ and X² are the same or different and include at least one functional group for binding to at least one of a phospholipid, a glycolipid, and a sterol;

wherein m is an integer from 0 to 30;

wherein n is an integer from 0 to 30;

wherein w is an integer from 2 to 1000; and

wherein a lipid bound to the compound is adapted for incorporation into liposomes or micelles.

### BRIEF DESCRIPTION OF THE DRAWING

FIG. 1 shows a graph of HIV-induced syncytium formation as a percentage of untreated control versus concentration (micromoles per liter) of the unseparated 3:1 mixture of racemic (Z)-(±)- and racemic (E)-(±)-ajoenes (♦), (Z)-(+)-ajoene (+), racemic (E)-(±)-ajoene (o), and (Z)-(−)-ajoene (x) (curves 1 through 4, left to right, respectively).

### DETAILED DESCRIPTION

It has been discovered that the activity of ajoene as a chiral integrin modulator (CIM) may be traced to its ability to form in vivo one or a plurality of active moieties having the formula:

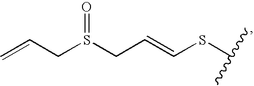

which include the (2E)-(+)-, (2E)-(−)-, (2Z)-(+)-, and (2Z)-(−) isomers. The active moiety shown above includes a 1,5-dithiaocta-2,7-diene-5-oxide-1-yl fragment (hereinafter DODOyl), which is incorporated into integrin modulators in accordance with the present invention, as further described below.

In accordance with this discovery, integrin modulators have been discovered that have a formula:

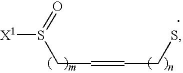

wherein:

X¹ is selected from the group consisting of hydrogen, an alkyl, an alkenyl, an alkynyl, an aryl, an aryl substituted with one or more —NO₂ groups, an aryl substituted with one or more lower alkyls, a group of formula RO—, a group of formula RC(O)—, a group of formula RC(O)O—, a group of formula ROC(O)—, a group of formula (R)₂N—, a group of formula RC(O)N—, a group of formula R(NH)C(O)—, a group of formula RN═N—, a group of formula RS—, a group of formula RSO₂—, a group of formula RS(O)—, a group of formula RSC(O)—, a group of formula RSO₂O—, a group of formula RS(O)O—, a halogen atom, a nitroso group, a furanose unit, a pyranose unit, a combination of furanose units, a combination of pyranose units, a combination of furanose and pyranose units, and combinations thereof;

R is selected from the group consisting of a hydrogen, a lower alkyl, a lower alkenyl, an aryl, an aryl substituted with one or more lower alkyls, S-cysteinyl, peptidyl, an alkylphosphoglyceryl, an alkenyiphosphoglyceryl, or an acylphosphoglyceryl;

m is an integer from 0 to 30; and

n is an integer from 0 to 30.

In further accordance with this discovery, integrin modulators have been discovered that have a formula:

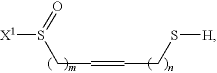

wherein X¹, R, m, and n are as defined above for the corresponding thia radical.

In further accordance with this discovery, integrin modulators have been discovered that include compounds capable of forming an active moiety analogous to the DODOyl fragment shown above, as well as products formed therefrom (e.g., by bond-formation and/or rearrangement). Integrin modulators embodying features of the present invention include DODOyl compounds and derivatives thereof, hereinafter referred to collectively as DODOyl-derived compounds (DDCs), which have a general formula:

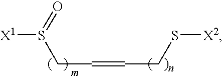

wherein:

X¹ and X² are the same or different and are each independently selected from the group defined above for the X¹ portion of the corresponding thia radical;

R is independently in each occurrence selected from the group defined above for the R group of the corresponding thia radical; and

m and n are as defined above for the corresponding thia radical;

providing that when X¹ is CH₂═CH—CH₂—, m is 1, and n is 0, X² is not CH₂═CH—CH₂—S— or (NH₂)—CH(CO₂H)—CH₂—S—.

In the above-described formula, m can also be an integer from 0 to about 3, and n can also be an integer from 0 to about 3. In a first series of presently preferred DDCs embodying features of the present invention, m=1 and n=0. In this first series, it is presently preferred that X¹ is alkenyl. Moreover, it is presently preferred that at least one double bond in the alkenyl X¹ fragment be allylic to the sulfoxide group in order to resemble the structure of the terminal double bond allylic to the sulfoxide group in ajoene. One presently preferred alkenyl group corresponds to X¹ is allyl. In a second series of presently preferred DDCs embodying features of the present invention, m=0 and n=1.

When X¹ is allyl, m=1, and n=0, the formula above corresponds to the prototypal DODOyl substituted compounds, which constitute a presently preferred DDC subgroup in accordance with the present invention. X² in this subgroup may correspond to any of the groups described above with the exception of CH₂═CH—CH₂—S— and (NH₂)—CH(CO₂H)—CH₂—S—. As described above, it is presently preferred that the X¹ portion of DDCs in accordance with the present invention be structurally analogous to the allyl group attached to the sulfoxide in ajoene. However, since the X² portion at the other end of the DDC may be cleaved to generate a thia moiety, as described above, a wide array of functionalities may be included therein.

Yet another distinct DDC subgroup embodying features of the present invention includes compounds which share two common characteristics: (a) the polymeric nature of X² and (b) multiplicity of the active chiral moieties attached to a single X². These compounds have a general formula:

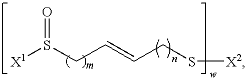

wherein X¹, m, and n are as defined above for the corresponding thia radical;

wherein X² is a polysaccharide, polypeptide, or other suitable substrate; and

w is an integer from 7 to 1000.

Polymeric DDCs embodying features of the present invention may also have a formula:

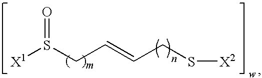

wherein X¹ and X² may be the same or different and are preferentially selected from functionalities enabling introduction of the chiral moiety into a phospholipid, glycolipid, or sphingolipid; wherein m and n are as defined above for the corresponding thia radical; and wherein w is an integer from 7 to 1000. Incorporation of such lipids into liposomes or micelles results in a subgroup of DDCs that are both polymeric (i.e., each liposome carries a desired amount of DDCs) and particulate (i.e., suspension of liposomes).

In addition, polymeric DDCs embodying features of the present invention may also have a formula:

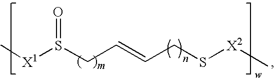

wherein X¹ and X² are the same or different and comprise at least one functional group configured for participation in a polymerization reaction;

wherein m is an integer from 0 to 30;

wherein n is an integer from 0 to 30; and

wherein w is an integer from 2 to 1000.

Additional polymeric DDCs embodying features of the present invention may have a formula:

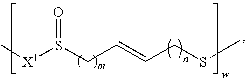

wherein X¹ comprises at least one functional group configured for participation in a polymerization reaction;

wherein m is an integer from 0 to 30;

wherein n is an integer from 0 to 30; and

wherein w is an integer from 2 to 1000.

It is understood that any DDC embodying features of the present invention includes the E and the Z stereoisomers (generated by the geometry of the central double bond), each having the R and the S configurations (generated by the presence of the sulfoxide functionality). The notations (E, R), (E, S), (Z, R), and (Z, S) are used herein to denote geometric isomers and enantiomers of DDCs embodying features of the present invention. Where applicable, as described herein, the (−) sign below the sulfur atom denotes the levorotatory or (−) enantiomer, and the (+) sign below the sulfur atom describes the dextrorotatory or (+) enantiomer. As will be understood by those of ordinary skill in the art, the optical rotation designations + and − are determined on a case-by-case basis for specific isomers using, for example, a polarimeter. It is to be understood that DDCs in accordance with the present invention may include one or more additional double bonds and/or additional chiral centers in the X¹ and/or X² portions thereof. Accordingly, when the E/Z designations are used herein to describe DDCs in accordance with the present invention, it is to be understood that these designations are used in reference to the geometry of the central double bond drawn in the general formulae (i.e., other double bonds in the molecule may have the same and/or different geometry as the central double bond). Similarly, when the R/S and/or (+)/(−)designations are used herein to describe DDCs in accordance with the present invention, it is to be understood that these designations are used in reference to the configuration of the central sulfoxide functionality drawn in the general formulae (i.e., additional chiral centers in the molecule may have the same and/or different chiral designation as the central sulfoxide group).

In view of the foregoing explanation, it is appreciated that, in one illustrative aspect, the compounds described herein can be mixtures containing all four enantiomers—that is, (E,R), (E,S), (Z,R), and (Z,S). In another illustrative aspect, the compounds described herein can be mixtures of two enantiomers having one double bond geometry or the other—that is, (E,R) and (E,S) or (Z,R) and (Z,S). In another illustrative aspect, the compounds described herein can be mixtures of two stereoisomers having the same sulfoxide chirality—that is, (E,R) and (Z,R) or (E,S) and (Z,S). In another illustrative aspect, the compounds described herein can be single enantiomers having one sulfoxide chirality and one double bond geometry—that is, (E,R), (E,S), (Z,R) or (Z,S). In another illustrative aspect, the compounds described herein can be mixtures of two enantiomers not matching each other in both the double bond geometry and the sulfoxide chirality—that is, (E,R) and (Z,S) or (E,S) and (Z,R). In yet another illustrative aspect, the compounds can be mixtures of any three out of the four possible enantiomers.

The present invention also provides methods of using the above-described DDCs.

In a first aspect, the present invention provides methods of modulating an integrin-mediated function of one or more cells using DDCs embodying features of the present invention.

In a second aspect, the present invention provides methods for the treatment of a variety of disorders, diseases and conditions. In particular, the present invention provides but is not limited to: (a) methods of treating or preventing a disorder, disease or condition in which one or more integrin-mediated functions play a role; (b) methods of treating or preventing thrombotic disorders and diseases/conditions arising therefrom (e.g., embolism, ischemia, infarction, etc.); (c) methods of treating or preventing inflammation and inflammatory diseases; (d) methods of treating, preventing, or inhibiting the transmission of viral infections; (e) methods of treating shock; (f) a method of treating arthritis; (g) methods of contraception; (h) methods of treating or suppressing adverse, undesirable or self-destructive immune responses, including but not limited to acute and chronic hypersensitivity reactions (such as anaphylaxis and allergy), transplant rejection, and graft-versus-host disease (GVHD); (i) methods of treating autoimmune diseases; (j) methods of inhibiting undesirable integrin-mediated cell-cell fusion; (k) methods of inhibiting the formation of lesions; (l) methods of treating psoriasis; (m) methods of treating atherosclerosis; (n) methods of treating diseases or conditions involving a plurality of integrin-dependent etiopathogenetic mechanisms; (o) methods of inhibiting the transfer of genetic material; (p) methods of treating cancer, preventing metastasis of tumors, or inhibiting certain (integrin-mediated) types of carcinogenesis; (q) methods of treating or preventing diseases involving both autoimmune and inflammatory conditions (e.g., diabetes, lupus, etc.); and the like.

The present invention further provides pharmaceutical compositions. The compositions include a DDC embodying features of the present invention and a pharmaceutically-acceptable carrier.

In addition, the present invention provides methods of treating an organ or a tissue thereof by contacting the organ or tissue with a DDC embodying features of the present invention. Such treatment improves the condition of the organ or tissue for subsequent use, as compared to organ or tissue which is not treated with a DDC in accordance with the present invention. In particular, an organ or tissue which is to be transplanted into a recipient may be treated with a DDC prior to, in the course of, and/or after harvesting, and/or prior to, in the course of, and/or after transplantation, and the chances of the tissue being successfully transplanted will be increased.

The present invention further provides kits for treating tissue. The kits comprise one or more DDCs embodying features of the present invention.

In addition, the present invention provides methods whereby the integrin-modulating activity of DDCs may be assessed. One such method involves VLA-4-mediated adhesion of enzyme-labeled PM1 cells to VCAM-1-coated artificial substrata. Specifically, the cells are exposed to isolated DDC enantiomers (or the vehicle thereof) and allowed to adhere or not adhere to immobilized VCAM-1. Thereafter, the adherent cells are lysed and, following addition of the substrate of the enzyme and incubation, the activity of the enzyme is measured spectrophotometrically. The value of this parameter, characterizing the number of the adherent cells, is inversely proportional to integrin-modulating activity of the compound. Another such method is based on the inhibition of HIV-mediated syncytium formation, a phenomenon known to depend on integrin-mediated functions. Yet another method is based on the inhibition of cell-to-cell HIV transmission, a process also dependent on integrin-mediated functions.

CIMs based on enantiomers of ajoene and derivatives thereof are described in U.S. patent application Ser. No. 10/332,545, filed Jan. 9, 2003, International Application No. PCT/US01/21826, filed Jul. 10, 2001, and U.S. Provisional Patent Application No. 60/217,651, filed Jul. 10, 2000. The entire contents of all of the above-identified applications are incorporated herein by reference, except that in the event of any inconsistent disclosure or definition from the present application, the disclosure or definition herein shall be deemed to prevail.

Throughout this description and the appended claims, the following definitions are intended.

The phrase “integrin modulator” refers to an agent that adjusts, varies, modifies, alters or affects an integrin-mediated function. As used herein, the phrase “integrin modulator” includes agents that act directly on integrins as well as agents that act indirectly on integrins (e.g., by acting on one or more species that themselves act on or in concert with integrins).

The term “racemic” is used to describe mixtures of optical isomers of a chemical compound (e.g., dextrorotatory and levorotatory), which may correspond to substantially equal amounts thereof or to a preponderance of one or the other. As used herein, the term “racemic” is also used to describe mixtures of E and Z isomers of a double bond-containing compound (i.e., mixtures of E and Z isomers), which may correspond to substantially equal amounts thereof or to a preponderance of one or the other.

The term “tissue” is used in reference to both a substantially complete organ (e.g., a heart) as well as to any portion thereof (e.g., a ventricle). Thus, references herein to methods of treating tissue are to be understood as including methods for the treatment of organs as well.

The phrase “furanose unit” refers to a five-membered cyclic form of pentose, hexose, or heptose, optionally modified with amino groups, sulfhydryl groups, or residues of acetic, phosphoric, and/or sulfuric acids.

The phrase “pyranose unit” refers to a six-membered cyclic form of pentose, hexose, or heptose, optionally modified with amino groups, sulfhydryl groups, or residues of acetic, phosphoric, and/or sulfuric acids.

The related terms “polymer,” “polymerization,” and “polymeric” refer to molecules, or to the preparation of molecules, containing two or more monomeric units. Thus, as used herein, the term “polymer” subsumes the term “oligomer.”

The term “alkyl” refers to a straight-chain or branched-chain hydrocarbon containing 1-30 carbon atoms, or a cyclic hydrocarbon containing 3-20 carbon atoms. The alkyl may be substituted or unsubstituted. The phrase “lower alkyl” refers a straight-chain or branched-chain alkyl containing 1-4 carbon atoms. Both terms include all possible isomers. Representative presently preferred alkyls for use in accordance with the present invention include but are not limited to methyl, ethyl, propyl, iso-propyl, n-butyl, iso-butyl, sec-butyl, tert-butyl, pentyl, iso-pentyl, neo-pentyl, n-hexyl, cyclohexyl, and the like.

The term “alkenyl” refers to a straight-chain or branched-chain hydrocarbon containing 2-30 carbon atoms, or a cyclic hydrocarbon containing 3-20 carbon atoms, and one or a plurality of double bonds. The alkenyl may be substituted or unsubstituted. The term includes all possible isomers. Representative presently preferred alkenyls for use in accordance with the present invention include but are not limited to vinyl, propenyl, iso-propenyl, and the like.

The term “alkynyl” refers to a straight-chain or branched-chain hydrocarbon containing 2-30 carbon atoms, or a cyclic hydrocarbon containing 3-20 carbon atoms, and one or a plurality of triple bonds. The alkynyl may be substituted or unsubstituted. The term includes all possible isomers. Representative presently preferred alkynyls for use in accordance with the present invention include but are not limited to ethynyl, 1-propenyl, 2-propenyl, and the like.

The term “aryl” refers to a group containing at least one aromatic ring, with phenyl being a presently preferred example. The aryl may be substituted or unsubstituted. Electron-withdrawing groups are presently preferred substituents. For example, the aryl may be preferably substituted with one or more —NO₂ groups, in which case it is preferably m-nitrophenyl, p-nitrophenyl, o-nitrophenyl, 3,5-dinitrophenyl, or 2,4-dinitrophenyl. The term aryl also includes arylalkyl groups that may optionally be substituted with one or more groups selected from alkyl and —NO₂. Representative presently preferred arylalkyls for use in accordance with the present invention include but are not limited to benzyl, o-tolyl, p-tolyl, m-tolyl, 3,5-xylyl, 2,6-xylyl, and the like. The term also includes polycyclic aromatic groups. Presently preferred polycyclic aromatic groups for use in accordance with the present invention contain the cyclopentane perhydrophenanthrene (steroid) backbone.

The term “halogen” refers to an atom in Group VIIB of the periodic table (e.g., fluorine, chlorine, bromine, iodine, astatine, etc.).

The term “peptidyl” refers to a straight-chain or a branched-chain S-cysteinyl peptide function.

The term “alkylphosphoglyceryl” refers to 1-O-alkyl-2-O-sn-glyceroyl 3-O-phosphate, 2-O-alkyl-1-O-sn-glyceroyl 3-O-phosphate, 1-O-alkyl-2-N-iminoglyceryl 3-O-phosphate, 2-O-alkyl-1-N-iminoglyceryl 3-O-phosphate, 1-O-alkyl-2-S-thiaglyceryl 3-O-phosphate, 2-O-alkyl-1-S-thiaglyceryl 3-O-phosphate, and the like. The prefix “alkyl” as applied to a phosphoglyceryl is to be understood as defined above, with presently preferred alkyls including but not limited to straight-chain or branched-chain hydrocarbons.

The term “alkenylphosphoglyceryl” refers to 1-O-alkenyl-2-O-sn-glyceroyl 3-O-phosphate, 2-O-alkenyl-1-O-sn-glyceroyl 3-O-phosphate, 1-alkenyl-1-hydroxy-2-N-iminoglyceryl 3-O-phosphate, 1-O-alkenyl-2-N-iminoglyceryl 3-O-phosphate, 2-O-alkenyl-1-N-iminoglyceryl 3-O-phosphate, 1-O-alkenyl-2-S-thiaglyceryl 3-O-phosphate, 2-O-alkenyl-1-S-thiaglyceryl 3-O-phosphate, and the like. The prefix “alkenyl” as applied to a phosphoglyceryl is to be understood as defined above, with presently preferred alkenyls including but not limited to straight-chain or branched-Chain hydrocarbons.

Unless noted otherwise, the variable “R” included in the following definitions refers to hydrogen or to any substituted or unsubstituted alkyl, alkenyl, alkynyl, or aryl moiety, wherein alkyl, alkenyl, alkynyl, and aryl are as defined above.

The term “acylphosphoglyceryl” refers to 1-O-acyl-2-O-sn-glyceroyl 3-O-phosphate, 2-O-acyl-1-O-sn-glyceroyl 3-O-phosphate, 1-O-acyl-2-N-iminoglyceryl 3-O-phosphate, 2-O-acyl-1-N-iminoglyceryl 3-O-phosphate, 1-O-acyl-2-S-thiaglyceryl 3-O-phosphate, 2-O-acyl-1-S-thiaglyceryl 3-O-phosphate, and the like. The prefix “acyl” as applied to a phosphoglyceryl refers to a carbonyl-containing moiety, such as RC(O)—.

Presently preferred groups having a formula RO— include but are not limited to hydroxy, methoxy, ethoxy, phenoxy, benzyloxy, 1-O-alkyl-2-O-sn-glyceroyl 3-O-phosphate (or 2-O-alkyl-1-O-sn-glyceroyl 3-O-phosphate), 1-O-alkenyl-2-O-sn-glyceroyl 3-O-phosphate (or 2-O-alkenyl-1-O-sn-glyceroyl 3-O-phosphate), 1-O-acyl-2-O-sn-glyceroyl 3-O-phosphate (or 2-O-acyl-1-O-sn-glyceroyl 3-O-phosphate), in which the phosphate group may be esterified (with an organic base, an alcohol, or a sugar), and the like.

Presently preferred groups having a formula RC(O)— for use in accordance with the present invention, include but are not limited to acetyl, formyl, benzoyl, and the like.

Presently preferred groups having a formula RC(O)O— for use in accordance with the present invention include but are not limited to formyloxy, acetoxy, and the like.

Presently preferred groups having a formula ROC(O)— for use in accordance with the present invention include but are not limited to methoxycarbonyl, ethoxycarbonyl, tert-butoxycarbonyl, benzyloxycarbonyl, and substituted phosphoglyceryls wherein R is selected from 1-O-alkyl-2-O-sn-glyceroyl 3-O-phosphate, 2-O-alkyl-1-O-sn-glyceroyl 3-O-phosphate, 1-O-alkenyl-2-O-sn-glyceroyl 3-O-phosphate, 2-O-alkenyl-1-O-sn-glyceroyl 3-O-phosphate, 1-O-acyl-2-O-sn-glyceroyl 3-O-phosphate, 2-O-acyl-1-O-sn-glyceroyl 3-O-phosphate (in each of which the phosphate group may be esterified with an organic base, an alcohol, or a sugar), and the like.

Presently preferred groups having a formula (R)₂N— for use in accordance with the present invention include but are not limited to amino, methylamino, ethylamino, phenylamino, dimethylamino, diethylamino, and the like.

Presently preferred groups having a formula RC(O)N— for use in accordance with the present invention include but are not limited to acetylamino, benzoylamino, and the like.

Presently preferred groups having a formula R(NH)C(O)— for use in accordance with the present invention include but are not limited to substituted phosphoglyceryls wherein R is selected from 1-O-alkyl-2-N-iminoglyceryl 3-O-phosphate, 2-O-alkyl-1-N-iminoglyceryl 3-O-phosphate, 1-O-alkenyl-2-N-iminoglyceryl 3-O-phosphate, 2-O-alkenyl-1-N-iminoglyceryl 3-O-phosphate, 1-O-acyl-2-N-iminoglyceryl 3-O-phosphate, 2-O-acyl-1-N-iminoglyceryl 3-O-phosphate (in each of which the phosphate group may be esterified with an organic base, an alcohol, or a sugar), and the like.

A subset of groups of the preceding formula includes functionalities wherein R is 1-alkenyl-1-hydroxy-2-N-iminoglyceryl 3-O-phosphate, with tetradeca-1-ene-1-yl being a presently preferred alkenyl and the phosphate group being optionally esterified with an organic base, an alcohol or a sugar. This subset corresponds to sphingolipid derivatives, as shown below (glycerol backbone is drawn bold):

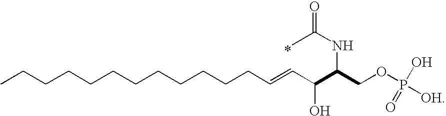

A presently preferred group having a formula RN═N— for use in accordance with the present invention includes but is not limited to phenylazo, and the like.

Presently preferred groups having a formula RS— for use in accordance with the present invention include but are not limited to ethylthio, S-cysteinyl, S-glutathionyl, 1-O-alkyl-2-S-thiaglyceryl 3-O-phosphate (or 2-O-alkyl-1-S-thiaglyceryl 3-O-phosphate), 1-O-alkenyl-2-S-thiaglyceryl 3-O-phosphate (or 2-O-alkenyl-1-S-thiaglyceryl 3-O-phosphate), 1-O-acyl-2-S-thiaglyceryl 3-O-phosphate (or 2-O-acyl-1-S-thiaglyceryl 3-O-phosphate), in which the phosphate group may be esterified (with an organic base, an alcohol, or a sugar), and the like.

A presently preferred group having a formula RSO₂— for use in accordance with the present invention includes but is not limited to methylsulfonyl, benzylsulfonyl, and the like.

A presently preferred group having a formula RS(O)— for use in accordance with the present invention includes but is not limited to methylsulfinyl, benzylsulfinyl, and the like.

A presently preferred group having a formula RSO₂O— for use in accordance with the present invention includes but is not limited to methylsulfonyloxy, benzylsulfonyloxy, and the like.

A presently preferred group having a formula RS(O)O— for use in accordance with the present invention includes but is not limited to methylsulfinyloxy, benzylsulfinyloxy, and the like.

Presently preferred groups having a formula RSC(O)— for use in accordance with the present invention include but are not limited to 1-O-alkyl-2-S-thiaglyceryl 3-O-phosphate (or 2-O-alkyl-1-S-thiaglyceryl 3-O-phosphate), 1-O-alkenyl-2-S-thiaglyceryl 3-O-phosphate (or 2-O-alkenyl-1-S-thiaglyceryl 3-0-phosphate), 1-O-acyl-2-S-thiaglyceryl 3-O-phosphate (or 2-O-acyl-1-S-thiaglyceryl 3-O-phosphate), in which the phosphate group may be esterified (with an organic base, an alcohol, or a sugar), and the like.

It has been found that Z(−)-4,5,9-trithiadodeca-1,6,11-triene-9-oxide is at least four times more active as an integrin modulator than other enantiomers, racemic E-ajoene, or the unseparated 3:1 mixture of racemic E and Z-ajoenes. The superior activity of one out of the four enantiomers correlates with the presence in the molecule of the following substructure:

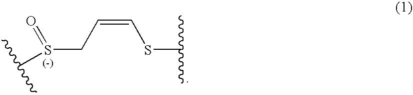

Chiral compounds containing the above-described core structure thus comprise a class of potent integrin modulators. Specifically, this class includes compounds of the formula:

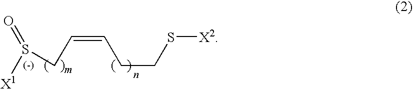

The formula (2) above corresponds to ajoene when X¹ and X² are both CH₂═CH—CH₂—, m is 1, and n is 0.

A mixture of four enantiomers, including a compound of formula (2) as one of the enantiomers, can be synthesized by methods well known in the art, such as those described in U.S. Pat. Nos. 4,643,994 and 4,665,088, in Block et al., J Am. Chem. Soc., 1986, 108, 7045, and in Sendl et al., Planta Med., 1991, 57, 361. The entire contents of all four documents are incorporated herein by reference, except that in the event of any inconsistent disclosure or definition from the present application, the disclosure or definition herein shall be deemed to prevail. Such a mixture, having a general formula (2a), can then be separated as described below to obtain a desired compound, for example, (Z)-(−)-4,5,9-trithiadodeca-1,6,11-triene-9-oxide:

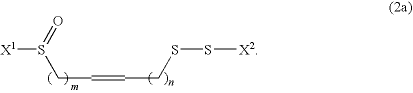

As further described below, compounds of formula (2a) may be cleaved at the disulfide bond and converted to DDCs in accordance with the present invention.

U.S. Pat. No. 4,665,088 describes the synthesis of (E,Z)-4,5,9-trithiadodeca-1,6,11-triene-9-oxide. Briefly, garlic is subjected to any convenient extraction procedure which acts to isolate the allicin component of garlic so that this component can be thereafter dissolved in an appropriate lower alkanol for a time and at a temperature sufficient to form (E,Z)-4,5,9-trithiadodeca-1,6,11-triene-9-oxide. To obtain higher yields, the garlic should be freshly cut, chopped or ground. Whole garlic cloves reduce yield, but can be satisfactorily used. The garlic pieces are blended with a volatile, water-miscible organic solvent such as a lower alkanol, ether, or acetone and allowed to sit for several hours or days. The particulate material is usually removed prior to further processing. Vacuum concentration of the liquid and extraction of the aqueous residue with an appropriate solvent, such as diethyl ether, appears to increase the yield significantly. The extracted aqueous residue can be washed several times with water, dried and evaporated to increase the purity of the oily allicin residue. The oily residue product is then dissolved in a volatile organic solvent, such as acetone or a lower alkanol in mixture with water (10-90%), and maintained at a temperature of from about −40° C. to a temperature less than about the reflux temperature of the organic solvent in mixture with the water. Generally, the higher the temperature, the lower the amount of time the mixture should be maintained at that temperature. It is generally desirable to adjust temperature to achieve a maintenance time of several hours, usually from about 10-72 hours. (E,Z)-4,5,9-trithiadodeca-1,6,11-triene-9-oxide can also be prepared as described below.

U.S. Pat. No. 4,643,994 describes the synthesis of compounds of formula (2a)—that is, mixtures of enantiomers, which contain several compounds that fall within formula (2). This patent also sets forth a general synthetic method that can be used for preparing compounds of formula (2a). Briefly, an appropriate disulfide radical having the formula:

X¹—S—S-Q (3)

wherein Q is —(CH₂)ₘ—CH═CH—(CH₂)ₙ—H, and m, n, and X¹, may be as defined above for the DDCs embodying features of the present invention, is treated with an oxidizing agent, preferably in the presence of a solvent, and preferably at a temperature of from about −40° C. to about 65° C. to produce a thiosulfinate of the formula:

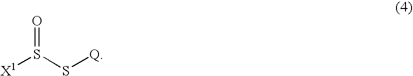

The thiosulfinate is then heated, typically refluxed, in the presence of an appropriate solvent, preferably a 60:40 organic solvent:water mixture to form a trithio oxide of the formula:

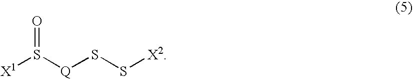

Typically, the reaction which causes the formation of the trithio oxide of formula (5) also causes the formation of minor products wherein each X¹ or Q can be Q or X¹, respectively. If a mixture of disulfides or thiosufinates is used as the starting compounds, the product will be a further mixture of products. The mixture of products can be separated at this point in the process by various means, such as extraction, or the mixture can be maintained as such through the next step(s). The compound (5) can be used directly to make DDCs or can be further oxidized to produce additional compounds of formula (2). Treatment with a stoichiometric amount (or a slight excess) of oxidizing agent forms compounds of formula:

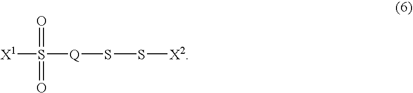

Treatment with further oxidizing agents at −30° C. to 40° C. produces compounds of formula:

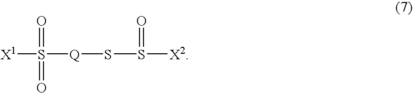

Continued treatment with an oxidizing agent produces compounds of formula:

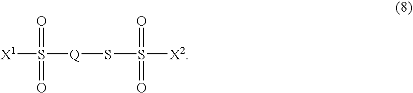

Each of compounds (7) and (8) can be reacted with a thiol of the formula X₁SH, or an alkali metal salt thereof, wherein X₁ is as defined above, to produce further compounds having different selected substituents as the X₁ moiety. These compounds can be purified by various means, including extraction, and used to make DDCs. The DDCs can be purified from an isomeric mixture by high performance liquid chromatography (HPLC) on columns packed with sorbents that are capable of separating individual enantiomers from mixtures thereof (cf. Examples below).

As described above, the integrin-modulating activities of ajoene, including the activity of Z(−)-ajoene as a CIM, may be traced to the ability of ajoene and its enantiomers to form in vivo enantiomeric (2E)-(+)-, (2E)-(−)-, (2Z)-(+)-, and (2Z)-(−)-DODOyl substituted compounds of the formula:

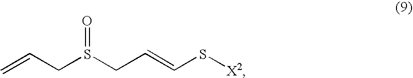

wherein X² may correspond to a free radical or a moiety attached thereto.

Thus, DDCs embodying features of the present invention include derivatives of compounds of formula (9), and have a general formula:

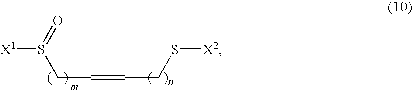

wherein X¹ and X² are as defined above.

Compounds of formula (10) may be accessed from compounds of formula (2a) by various methodologies well known in the art. For example, the disulfide bond of (2a) may be cleaved with a mild reducing agent (e.g., zinc and dilute acid; Ph₃P and H₂O; heating with alkali; etc.) to form the two corresponding thiols. The DDC fragment (i.e., formula (10) in which X² is hydrogen) may be elaborated to introduce various other X² groups to form DDCs in accordance with the present invention (e.g., by nucleophilic substitution reactions known in the art, wherein a leaving group in the X² residue to be attached is displaced by the thiol sulfur atom, in the DDC fragment, or a salt thereof).

Presently, a particularly preferred DDC subgroup is constituted by the prototypal DODOyl substituted compounds of formula (9)—that is, compounds of formula (10), wherein X¹=allyl, m=1, and n=0.

Yet another distinct DDC subgroup includes compounds which share two common characteristics: (a) the polymeric nature of X² and (b) multiplicity of the active chiral moieties attached to a single X². These compounds have a general formula:

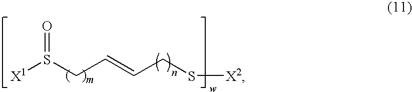

wherein X² is a polysaccharide, polypeptide, or other suitable substrate;

X¹, m, and n are the same as defined for formula (10) above; and w is an integer from 7 to 1000.

A compound of formula (11), wherein X² is a polysaccharide, is a polymer of furanose and/or pyranose units carrying N-, C-, or S-linked DDC functions, or derivatives thereof.

A compound of formula (11), wherein X² is a polypeptide, is a synthetic structure the cysteine residues of which carry S-linked DDC functions, or derivatives thereof.

Polymeric DDCs may also have the formula:

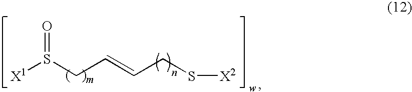

wherein X¹ and X² may be different and are preferentially selected from functionalities enabling introduction of the chiral moiety into a phospholipid, glycolipid, or sphingolipid (e.g., allyl, allylthio, substituted furanose/pyranose units, polycyclic aryls, and substituted phosphoglyceryls), with subsequent incorporation of these lipids into liposomes or micelles.

A typical compound of formula (12), therefore, is a liposomal preparation containing a lipid modified, via S-alkylation, N-alkylation, O-alkylation, or O-acylation, with a DDC function or its derivative.

Polymeric DDCs may also have the formula:

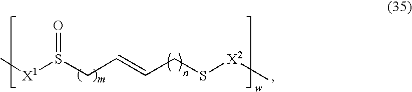

wherein X¹ and X² are the same or different and comprise at least one functional group configured for participation in a polymerization reaction;

wherein m is an integer from 0 to 30;

wherein n is an integer from 0 to 30; and

wherein w is an integer from 2 to 1000.

Polymeric DDCs may also have the formula:

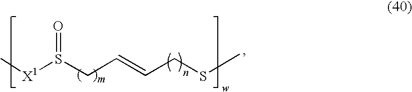

wherein X¹ comprises at least one functional group configured for participation in a polymerization reaction;

wherein m is an integer from 0 to 30;

wherein n is an integer from 0 to 30; and

wherein w is an integer from 2 to 1000.

It is understood that any DDC of formulae (10)-(12), (35), and (40) includes the E and the Z stereoisomers (generated by the geometry of the central double bond), each having the R and the S configurations (generated by the presence of the central sulfoxide functionality). It is appreciated that, in one illustrative aspect, the compounds described herein can be mixtures containing all four enantiomers—that is, (E,R), (E,S), (Z,R), and (Z,S). In another illustrative aspect, the compounds described herein can be mixtures of two enantiomers having one double bond geometry or the other that is, (E,R) and (E,S) or (Z,R) and (Z,S). In another illustrative aspect, the compounds described herein can be mixtures of two stereoisomers having the same sulfoxide chirality that is, (E,R) and (Z,R) or (E,S) and (Z,S). In another illustrative aspect, the compounds described herein can be single enantiomers having one sulfoxide chirality and one double bond geometry that is, (E,R), (E,S), (Z,R) or (Z,S). In another illustrative aspect, the compounds described herein can be mixtures of two enantiomers not matching each other in both the double bond geometry and the sulfoxide chirality that is, (E,R) and (Z,S) or (E,S) and (Z,R). In yet another illustrative aspect, the compounds can be mixtures of any three out of the four possible enantiomers.

In accordance with the present invention, safe and effective doses of the DDCs may inhibit the progression of an HIV infection in a patient or the infection of an uninfected patient by HIV. While Z(−)-ajoene is one such inhibitor, other DDCs may also be used.

One of the characteristics of the inhibition of HIV infection is the diminution of the formation of HIV-induced syncytia, in which HIV target cells, such as lymphocytes and monocytes, fuse together to form giant, multinucleate cells. Transfer of genetic material between cells may, thereby, also be inhibited by DDCs, which inhibit fusion with cell membranes.

Additionally, because DDCs may modulate pertinent integrin-mediated activity, DDCs may inhibit the entry of the infective HIV material into its target cells, including CD4-negative cells, both virus-to-cell and cell-to-cell entry, and the production of HIV and other viruses by the infected cells. For these purposes, DDCs in accordance with the present invention may preferably be administered in a sufficient dose to provide a concentration approaching or exceeding 5 micromoles per liter of a patient's blood plasma, although lesser concentrations may also be effective and may also be effective in sustained (i.e., chronic) administration. This dose may be effective when the DDCs are used alone or in bi- or multi-therapy addressing different disease parameters (e.g., the function of viral enzymes).

In addition to infections caused by HIV and other viruses of the Retroviridae family, DDCs embodying features of the present invention may be used locally or systemically to inhibit or prevent the transmission, in vivo and in vitro, of other viruses infecting humans and other animals. In particular, DDCs embodying features of the present invention may be used to treat or prevent infections caused by viruses, the transmission of which involves fusion of at least a part of the virus with the membrane of the target cell that is to be infected. Such viruses include all enveloped viruses and other viruses that infect cells in this manner. The enveloped viruses include the Retroviridae, Herpesviridae (e.g., herpes simplex virus 1 [HSV-1], HSV-2, varicella zoster, Epstein-Barr virus, and cytomegaly virus), Hepadnaviridae (e.g., hepatitis B), Flaviviridae (e.g., yellow fever virus and hepatitis C virus), Togaviridae (e.g., rubivirus, such as rubella virus, and alphavirus), Orthomyxoviridae (e.g., influenza virus), Paramyxoviridae (e.g., measles, parainfluenza, mumps and canine distemper viruses), Poxviridae (e.g., variola virus and vaccinia virus), and Rhabdoviridae (e.g., rabies virus). Other viruses that infect cells by fusing with the membrane include Papovaviridae (e.g., papillomavirus), Picornaviridae (e.g., hepatitis A virus and poliomyelitis virus), Rotaviridae, and Adenoviridae. In addition to inhibiting or preventing virus-to-cell entry, DDCs may also inhibit or prevent cell-to-cell transmission of viruses (e.g., by inhibiting or preventing syncytia formation or by inhibiting or preventing intercellular viral transfer between cells in contact or close proximity) and the production of viruses by infected cells.

DDCs embodying features of the present invention may also serve as agents that inhibit the adhesion, migration (e.g., chemotaxis or other infiltration of the tissue), and aggregation of various cell types and lines, including blood platelets and neutrophils. Thus, DDCs in accordance with the present invention may exhibit benefit as agents for the treatment of pathologies derived from adhesion, migration, and aggregation of these and other cells, including thrombosis and various types of inflammation.

Thrombosis is defined as blockage of blood vessel(s) by thrombi (i.e., clots formed from fibrin and platelet aggregates) deposited on the inner surface of the vessel. Thrombi form in arteries (e.g., damaged as a result of a disease) or in veins (e.g., due to lengthy immobilization). If a thrombus or a blood clot is dislodged and moves through the bloodstream to create an obstruction outside the place of its formation, it becomes an embolus (hence the terms “thromboembolism” and “thromboembolic disease”). Thrombosis or thromboembolism of coronary arteries can cause heart attacks and myocardial infarction; the same processes in brain arteries cause stroke. Inhibition of platelet aggregation by DDCs in accordance with the present invention may, therefore, arrest thrombosis at early stages, precluding the development of thrombotic and thromboembolic diseases.

Inflammation, a pathological process inherent in a variety of distinct diseases and illnesses, is defensive in nature, but potentially dangerous if uncontrolled. When viewed at the “whole body” level, an inflammation is most frequently characterized by several localized manifestations (indices), including hemodynamic disorders (e.g., hyperemia and edema), pain, temperature increment, and functional lesion. These inflammatory phenomena are underlain by events at the cellular and molecular levels. At the cellular level, inflammation is characterized by leukocyte extravasation (a process involving adhesion of leukocytes to the endothelium of the vessel wall and migration into tissue where they may phagocytose bacteria, viruses, and cell debris) and platelet aggregation (a mechanism, inter alia, whereby the spread of the infection is prevented). At the molecular level, inflammation is characterized by activation of at least three plasma defense systems (complement, kinin, and coagulation/fibrinolysis cascades) and by synthesis of cytokines and eicosanoids. When inflammation becomes generalized (e.g., as in the case of shock), various indices of inflammation occur systemically throughout the entire organ/organism. In cases of shock, platelets and leukocytes (principally neutrophils) aggregate in the blood vessels, leading to the development of a clinical condition known as multiple organ failure. The primary organ affected in shock patients is commonly the lung. Lung failure, or adult respiratory distress syndrome (ARDS), a destructive inflammation resulting from adhesion, aggregation, and degranulation of activated neutrophils in the pulmonary microvasculature, may be the main cause of death in patients suffering shock. DDCs embodying features of the present invention may thus counteract at least part of the effects of shock, whether arising, for example, from sepsis, anaphylaxis, blood loss, or from other precipitating events.

DDCs embodying features of the present invention may also be administered in effective dosages to suppress many other acute inflammatory processes, such as those associated with peritonitis, meningitis, and ischemia-reperfusion. Ischemia-reperfusion injury occurs (e.g., in heart, brain, kidney, liver, lung, intestinal tract, or any limb) when blood supply is abruptly stopped (ischemia) and then resumed (reperfusion) after a short period.

With the onset of ischemia and the decrease in the perfusion pressure, neutrophils are retained in the capillaries. As the ischemia progresses, cytokines (and other chemoattractants) are released into the capillary lumina in regions of the tissue where the blood flow blockage has occurred, increasing the adhesiveness of the retained neutrophils to the endothelium and to each other. Aggregates of neutrophils thus formed obstruct postcapillary venules (“no-reflow” or “no-washout”) and attenuate the restoration of the blood flow in the affected region, precluding its reoxygenation and extending the area of ischemia. Activated neutrophils trapped in the capillaries also release hydrolytic enzymes and reactive oxygen species (i.e., the armamentarium ordinarily used to defend the host against microorganisms), producing a destructive inflammation.

Restoration of the blood flow, however, further augments the severity of the inflammation thus developed. Neutrophils arriving to the previously ischemic region are activated (by chemoattractants and/or products released by the trapped neutrophils) and recruited into the tissue, where the defensive machinery of the cells is once again used against the host (secondary injury). Ischemia-reperfusion injury can also be generalized, for example, in the case of resuscitation after hemorrhagic shock (A. Mazzone et al., “Leukocyte CD 11/CD18 Integrins: Biological and Clinical Relevance,” Haematologica 1995, 80, 161; W. H. Reinhart, “Hemorheology: Blood Flow Hematology,” Schweiz. Med. Wochenschr. 1995, 125, 387).

DDCs embodying features of the present invention may be potent inhibitors of adhesive interactions for other cells, such as lymphoid cells. Adhesion of lymphocytes to each other and to nonlymphoid cells is prerequisite to the development of any immune response. Thus, DDCs in accordance with the present invention may serve as agents for the prevention, treatment, and control of adverse, undesirable, and self-destructive immune responses.

One group of such immunopathologies includes diseases stemming from divers allergic reactions (e.g., delayed type hypersensitivity, Arthus reaction, and anaphylaxis). Allergy is an anomalous immune response to antigen challenge, characterized by recruitment of specific leukocyte subsets (e.g., cytotoxic lymphocytes and/or eosinophils) to the tissue, resulting in inflammation. Development of allergic inflammation is the main component in the pathogenesis of many diseases and illnesses, including, for example, asthma, eczema, purpura pigmentosa chronica, various vasculitides, and hay fever, in addition to those described above. DDCs in accordance with the present invention may serve to control these diseases and illnesses.

Allograft rejection is another example of an undesirable immune response, in which the transplanted organ is recognized by the immune system as a foreign body (“non-self”) and attacked in sequence by cytotoxic lymphocytes and phagocytes recruited from the circulation. This inflammatory response results in progressive disruption of the tissue, including graft necrosis. DDCs embodying features of the present invention may be used to prevent cell recruitment into transplanted tissue and thereby prolong graft survival by reducing both acute and chronic aspects of rejection.

Moreover, the transplanted organ also contains lymphocytes, which, in turn, recognize their new environment as “non-self.” The immune response initiated by these donor lymphocytes in the body of the recipient produces a condition known as graft-versus-host disease (GVHD), which can lead to injury, both acute and chronic. DDCs embodying features of the present invention may contribute to the control of both acute and chronic GVHD.

Any method of treatment that suppresses both rejection and susceptibility to viruses (which, like cytomegaly virus, frequently contaminate the transplanted organs and decrease the probability of their engraftment) will have an extra benefit to the graft recipient. As described above, DDCs embodying features of the present invention may exert pronounced antiviral effects, in addition to being potent anti-inflammatory agents. Thus, administration of DDCs to patients undergoing organ transplantation offers much promise as a novel therapeutic approach to the prevention of rejection. Self-destructive responses are caused by the failure of the immune system to distinguish “self” from “non-self.” This group of immunopathologies includes a wide variety of diseases (herein termed collectively “autoimmune diseases”), including but not limited to rheumatoid arthritis, systemic lupus erythematosus, Sjogren's syndrome, multiple sclerosis, insulin-dependent diabetes mellitus, glomerulonephritis, Graves disease, Hashimoto's thyroiditis, and vasculitides. Other conditions and diseases may also fall into this category (e.g., see the description of psoriasis below) or include a component that does so (e.g., chronic viral diseases stimulating an autoimmune response). In spite of pronounced differences in the clinical picture of the various autoimmune diseases, the underlying mechanisms involve, in every case, undesirable recruitment of leukocytes to organs/tissues affected, resulting in destructive inflammation. DDCs embodying features of the present invention may be used to reduce or prevent this cellular recruitment and thereby suppress the abnormal immune response. Accordingly, DDCs in accordance with the present invention may be used to treat autoimmune diseases.

Without wishing to be bound to a particular theory or in any way limit the scope of the appended claims or their equivalents, it is presently believed that the beneficial effects of DDCs in accordance with the present invention are achieved because these substances are modulating integrin-mediated functions. As used herein, “modulate” means to affect the development or expression of modalities normally characterizing cellular activities and/or functions mediated by integrins. A modulating agent may act on an integrin directly, for example, by binding to or interacting with a portion of at least one subunit (alpha or beta) of the integrin. The agent may also act in some other fashion that is not considered direct, for example, through any of the various cellular substances and structures which ordinarily interact with or enable the participation of specific integrins, alone or in combination. These substances and structures include but are not limited to transmembrane proteins (e.g., integrins themselves and integrin-associated proteins), membrane phospholipids, intracellular molecules with messenger-like function (e.g., integrin-modulating factor), enzymes, lipid rafts, and regulatory and signaling proteins. Thus, for example, a modulation may result from alteration in integrin expression, activation, conformation, association or disassociation of the alpha and beta integrin subunits (or any parts thereof) affecting integrin clustering or clustering abilities, association or disassociation of integrin clusters (and of clusters formed by integrins with other proteins), or from the variations of integrin-cytoskeleton associations, although modulation may also occur from other types of effects. The functions of integrins, as defined herein, are interrelated and include but are not limited to, inter alia, signaling, adhesion, fusion, internalization, cellular conformation, regulation of lipid raft distribution, and microtubule stabilization (For recent research vis-à-vis several of these functions, see: J-L Guan, “Integrins, Rafts, Rac, and Rho,” Science, 2004, 303, 773-774; A. F. Palazzo et al., “Localized Stabilization of Microtubules by Integrin- and FAK-Facilitated Rho Signaling,” Science, 2004, 303, 836-839; and M. A. Del Pozo et al., “Integrins Regulate Rac Targeting by Internalization of Membrane Domains,” Science, 2004, 303, 839-842).

As a result of their ability to modulate activities in which integrins participate, DDCs embodying features of the present invention may be used to treat a plurality of diseases or conditions that involve undesirable integrin-mediated functions as a mechanism, including those described above. For instance, DDCs in accordance with the present invention may be used to inhibit virus-cell fusion, to modulate molecular exchanges arising from cell-to-cell cytoplasmic interactions, and/or to otherwise modulate undesired cell-cell fusion.

Undesired cell-cell fusion may include, for example, cell-cell fusion (transitory or permanent) that results in the transfer of viral genetic material; cell-cell fusion that results in the formation of multinucleate cells (e.g., syncytia, giant cells, and osteoclasts); undesired fertilization of eggs by sperm; and the formation of multinucleate germinal cells (syncytiotrophoblast).

Thus, DDCs embodying features of the present invention may be used as contraceptives, being administered per vaginam (topically), per os, or in any other appropriate way, when used for this purpose. DDCs in accordance with the present invention may also prevent conception, however, at the stage of embryo implantation. For example, DDCs may prevent the initial adhesion of the blastocyst to the endometrium and the migration of cytotrophoblasts through the maternal epithelium (i.e., processes similar to certain steps in the leukocyte extravasation cascade and tumor cell metastasis).

Furthermore, DDCs embodying features of the present invention may be capable of inhibiting cytotrophoblast invasion, a process differing from extravasation in that it goes from the tissues to the vascular lumen and in that the invading cells cross the blood-tissue barrier from outside of the vessel (reverse diapedesis). For example, the production of proteolytic enzymes that are used by cytotrophoblasts to penetrate the basement membrane is governed by integrin outside-in signaling. Modulation of the signaling function of integrins by DDCs in accordance with the present invention may either completely prevent the production of the requisite enzymes or attenuate it to an extent precluding invasion. A related mechanism underlies the ability of DDCs to block angiogenesis, preventing the blood supply to the fetal tissue. Thus, DDCs embodying features of the present invention may be used as effective emergency contraceptives to prevent unwanted pregnancy or to interrupt it at an early stage. There are additional mechanisms whereby DDCs, when desired, can exert contraceptive effects. For example, they can prevent the chemotactic response of sperm in the vaginal environment (a specific case of cell homing) and sperm interactions with the epithelium of the female genital tract. Moreover, DDCs embodying features of the present invention may be administered to males to modulate integrin-mediated functions in sperm precursors and other testicular or epididymal cells, thereby interfering with the maturation processes and resulting in the production of fertilization-incompetent gametes or inhibition of fertilization-competent gametes.

The development of major bone diseases, including osteoporosis, is underlain by excessive bone resorption. This fundamental function is performed by osteoclasts. Osteoclasts are multinucleate bone cells formed by fusion of mononuclear progenitors called preosteoclasts. The regulation of osteoclast formation may be achieved by agents acting at various levels of osteoclast formation, including preosteoclast fusion (Zaidi et al., “Cellular Biology of Bone Resorption,” Biol. Rev. 1993, 68, 197). DDCs embodying features of the present invention may regulate bone resorption because they inhibit the fusion of preosteoclasts, necessary for the formation of osteoclasts.

Granulomas are characteristic of chronic inflammatory lesions, such as those found in tuberculosis and other chronic infections. Granulomas are also present in sarcoidosis, a chronic, systemic inflammatory disease of unclear etiology. Granulomas present in cases of chronic infection and in sarcoidosis contain a large number of multinucleate giant cells formed by the fusion of macrophages. Other diseases associated with the formation of multinucleate cells include but are not limited to Crohn's disease, Langerhans cell histiocytosis, and giant cell arteriitis. DDCs embodying features of the present invention may be used to inhibit the formation of these giant multinucleate cells with beneficial therapeutic effects.

Excessive formation of fibrous interstitial tissue (i.e., fibrosis, or sclerosis including, for example, multiple sclerosis and scleroses of specific organs, such as sclerosis of the liver) is characteristic of certain diseases (such as scleroderma and idiopathic pulmonary fibrosis) and an outcome of chronic inflammatory processes (e.g., glomerular fibrosis). The development of fibrotic lesions and progression of fibrosis, associated with these conditions, diseases, and illnesses, has been linked to abnormal integrin expression and altered cell adhesion patterns. DDCs embodying features of the present invention may, therefore, be used for treatment of fibrotic lesions, including the formation of keloid (scar tissue). Lesions observed in skin diseases and illnesses of diverse origin, such as lichen planus, urticaria, dermatofibroma, psoriasiform dermatitides, and keratoses, are characterized by aberrant integrin expression. DDCs in accordance with the present invention may serve as agents for the symptomatic treatment of these diseases, being administered topically, intradermally, and subcutaneously at the site of lesions, or in any other appropriate way, when used for this purpose.

Another disease characterized by the formation of cutaneous lesions is psoriasis. Although the etiology of psoriasis attends further elucidation (several viruses and an autoimmune component could be involved), its pathogenesis is associated with abnormal expression of integrins in target tissue (e.g., in vascular cells, keratinocytes, and dendritic cells), proliferation of endothelial and epidermal cells, and an autoimmune component (recruitment of lymphocytes and macrophages to skin and joints). DDCs embodying features of the present invention may, therefore, be used to treat psoriasis in multiple respects.

As a result of their antiviral and anti-inflammatory activity, DDCs embodying features of the present invention may exhibit significant potency in the prevention and treatment of certain diseases with combined etiopathogenesis. As roughly elaborated herein, the term “etiopathogenesis” is used in reference to diseases for which no distinction can be drawn thus far as to etiology and pathogenesis. In addition to psoriasis, described above, a good example of such a disease is atherosclerosis. A variety of pathogens may participate in the development of atherosclerosis. One of the best studied viral contributors is cytomegalovirus, which induces a specific type of infection characterized by plaque formation along the blood vessels (J. L. Melnick et al., “Cytomegalovirus and Atherosclerosis,” BioEssays 1995, 17, 899). A prominent feature of atherosclerosis is the recruitment of monocyte-macrophages into atherosclerotic plaques, which is an integrin-mediated process. Also, proliferation of smooth muscle cells, which contributes to the formation of atherosclerotic lesions, is regulated by integrins. As described above, DDCs embodying features of the present invention may inhibit the transmission of viral infections virus-to-cell and cell-to-cell. Integrin-mediated adhesion and signaling may also be inhibited by DDCs. Thus, for multiple reasons, DDCs in accordance with the present invention may be used to treat diseases involving combinations of integrin-mediated etiopathogenesic factors, including those that are in part of viral nature.

Certain neurodegenerative disorders of unclear etiology (e.g., Alzheimer's disease and amyotrophic lateral sclerosis) involve autoimmune inflammation of nervous tissue as a pathogenetic mechanism. Thus, DDCs embodying features of the present invention may demonstrate significant potency in mitigating the symptoms of these diseases and slowing their progression. This conclusion is further supported by various studies which show that other anti-inflammatory treatments benefit Alzheimer's patients (P. L. McGeer et al., “The Inflammatory Response System of Brain: Implications for Therapy of Alzheimer's and Other Neurodegenerative Diseases,” Brain Res. Brain Res. Rev. 1995, 21, 195; J. C. Breitner et al., “Delayed Onset of Alzheimer's Disease with Nonsteroidal Anti-Inflammatory and Histamine H₂ Blocking Drugs,” Neurobiol. Aging 1995, 16, 523).

To treat or prevent any of these disorders, diseases or conditions, an effective amount of a DDC embodying features of the present invention may be administered to an animal in need thereof. Preferably, the animal is a mammal, such as a rabbit, goat, dog, cat, horse or human. Effective dosage forms, modes of administration, and dosage amounts may be determined empirically, and making such determinations lies well within the skill of the ordinary artisan. It is understood by those of ordinary skill in the art that the dosage amount will vary with the disorder, disease or condition to be treated or prevented, the severity of the disorder, disease or other condition, which integrin-mediated function(s) is (are) to be modulated, the route of administration, the rate of excretion, the duration of the treatment, the identity of any other drugs being administered, the age, size and species of animal, and like factors well known in the arts of medicine and veterinary medicine. Typically, a suitable daily dose of a DDC will be that amount of the compound which is the lowest dose effective to produce the desired effect. The effective daily dose of a DDC embodying features of the present invention may be administered as two, three, four, five, six or more sub-doses, administered separately at appropriate intervals throughout the day. An existing disorder, disease or condition treated with a DDC or combination of DDCs in accordance with the present invention may be reduced, inhibited, suppressed or eliminated or one or more symptoms of the disorder, disease or condition may be alleviated or eliminated.

DDCs embodying features of the present invention may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. For example, the topical application of DDCs to mucous membranes (in the form of creams, gels, suppositories, and other known means of topical administration) may be used to prevent HIV infection of mucosal cells, an important route of HIV transmission. In addition, intralymphatic administration of DDCs may be advantageous in preventing the spread of HIV within the body. Further, DDCs in accordance with the present invention may be administered in conjunction with other treatments for the disorder, disease or condition being treated with the DDC, such as other antiviral drugs, other contraceptives, and other anti-shock or anti-inflammatory drugs or treatments. DDCs in accordance with the present invention may be encapsulated or otherwise protected, against gastric or other secretions, if desired.

While it is possible for a DDC embodying features of the present invention to be administered alone, it is presently preferred that the DDC be administered as a pharmaceutical formulation (composition). The pharmaceutical compositions in accordance with the present invention may include one or more DDCs as an active ingredient in admixture with one or more pharmaceutically-acceptable carriers and, optionally, one or more other compounds, drugs, ingredients and/or materials. Regardless of the route of administration selected, the DDCs embodying features of the present invention may be formulated into pharmaceutically-acceptable dosage forms by conventional methods known to those of ordinary skill in the art (e.g., see: Remington's Pharmaceutical Sciences, Mack Publishing Co., Easton, Pa.). Pharmaceutical carriers are well known in the art (e.g., see: Remington's Pharmaceutical Sciences cited above and The National Formulary, American Pharmaceutical Association, Washington, D.C.) and include sugars (e.g., lactose, sucrose, mannitol, and sorbitol), starches, cellulose preparations, calcium phosphates (e.g., dicalcium phosphate, tricalcium phosphate and calcium hydrogenphosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly[orthoesters], and poly[anhydrides]), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, and the like.

Each carrier used in a pharmaceutical composition embodying features of the present invention should be “acceptable” in the sense of being compatible with the other ingredients of the formulation and not injurious to the animal. Carriers suitable for a selected dosage form and intended route of administration are well known in the art, and acceptable carriers for a chosen DDC, dosage form and method of administration can be determined using ordinary skill in the art. The pharmaceutical compositions embodying features of the present invention may, optionally, contain one or more additional ingredients and/or materials commonly used in pharmaceutical compositions. These ingredients and materials are well known in the art and include but are not limited to (1) fillers or extenders, such as starches, lactose, sucrose, glucose, mannitol, silicic acid or the like; (2) binders, such as carboxymethylcellulose, alginates, gelatin, polyvinyl pyrrolidone, hydroxypropylmethyl cellulose, sucrose, acacia or the like; (3) humectants, such as glycerol or the like; (4) disintegrating agents, such as agar-agar, calcium carbonate, potato or tapioca starch, alginic acid, certain silicates, sodium starch glycolate, cross-linked sodium carboxymethyl cellulose, sodium carbonate or the like; (5) solution retarding agents, such as paraffin or the like; (6) absorption accelerators, such as quaternary ammonium compounds or the like; (7) wetting agents, such as cetyl alcohol, glycerol monostearate or the like; (8) absorbents, such as kaolin, bentonite clay or the like; (9) lubricants, such as talc, calcium stearate, magnesium stearate, solid polyethylene glycols, sodium lauryl sulfate or the like; (10) suspending agents, such as ethoxylated isostearyl alcohols, polyoxyethylene sorbitol and sorbitan esters, microcrystalline cellulose, aluminum metahydroxide, bentonite, agar-agar, tragacanth or the like; (11) buffering agents; (12), excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, polyamide powder or the like; (13) inert diluents, such as water, other solvents or the like; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres; aluminum monostearate, gelatin, waxes or the like; (18) opacifying agents; (19) adjuvants; (20) emulsifying and suspending agents; (21), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, fatty acid esters of sorbitan or the like; (22) propellants, such as chlorofluorohydrocarbons or the like and volatile unsubstituted hydrocarbons, such as butane, propane or the like; (23) antioxidants; (24) agents which render the formulation isotonic with the blood of the intended recipient, such as sugars, sodium chloride or the like; (25) thickening agents; (26) coating materials, such as lecithin or the like; and (27) sweetening, flavoring, coloring, perfuming and preservative agents. Each such ingredient or material should be “acceptable” in the sense of being compatible with the other ingredients of the formulation and not injurious to the animal. Ingredients and materials suitable for a selected dosage form and intended route of administration are well known in the art, and acceptable ingredients and materials for a chosen DDC, dosage form and method of administration may be readily determined by those of ordinary skill in the art.

Pharmaceutical formulations in accordance with the present invention that are suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations can be prepared by methods well known in the art (e.g., by means of conventional pan-coating, mixing, granulation or lyophilization processes).

Solid dosage forms for oral administration (capsules, tablets, pills, dragees, powders, granules, and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient.

A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents.

Suspensions may contain suspending agents.

Formulations for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Formulations which are suitable for vaginal administration also include pessaries, tampons, creams, gels, pastes, foams or spray formulations containing such pharmaceutically acceptable carriers as are known in the art to be appropriate.

Dosage forms for topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams, and gels may contain excipients. Powders and sprays may contain excipients and propellants. Pharmaceutical compositions suitable for parenteral administrations may include one or more DDCs embodying features of the present invention in combination with one or more pharmaceutically-acceptable, sterile, isotonic, aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity may be maintained, for example, by the use of coating materials, by the maintenance of the required particle size in the case of dispersions, and by the use of surfactants. These compositions may also contain suitable adjuvants, such as wetting agents, emulsifying agents, and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it may be desirable to slow its absorption from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms maybe made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampoules and vials, and may be stored in a lyophilized condition requiring only the addition of the sterile liquid carrier, for example water for injection, immediately prior to use. Extemporaneous injection solutions and suspensions may be prepared from sterile powders, granules and tablets of a type described above.

The present invention further provides methods of modulating an integrin-mediated function of one or more cells. The methods include contacting the cell(s) with an amount of a DDC embodying features of the present invention that is effective to modulate the integrin-mediated function. Methods of contacting cells in vivo are the same as those described above for treating a disorder, disease or condition. Methods of contacting cells in vitro with compounds (e.g., placed in a solution, such as a cell culture medium, containing the compound) are well known in the art. Suitable conditions (time, temperature, concentrations, type of medium, etc.) are known or can be determined empirically as is well known in the art.

The present invention also provides methods of treating a tissue by contacting the tissue with a DDC embodying features of the present invention. Such treatment may improve the condition of the tissue for subsequent use, as compared to tissue which is not treated with a DDC. In particular, tissue which is to be transplanted into a recipient may be treated with a DDC, preferably before excision, or, if not, at the time of excision, and the chances of the tissue being successfully transplanted may be increased. The tissue to be treated may be any tissue. For example, the tissue may be an organ tissue (such as tissue from a heart, blood vessel, lung, liver, kidney, skin, cornea, or tissue from part of an organ, such as a heart valve) or a non-organ tissue (such as tissue from bone marrow, stern cells, or gametes). The tissue is treated by contacting it one or more times with an effective amount of a DDC embodying features of the present invention. Methods of contacting tissues with agents are well known in the art. For example, the contacting may be accomplished conveniently by rinsing or perfusing the tissue with, and/or submersing the tissue in, a solution of the DDC in a physiologically acceptable diluent. Contacting may also include perfusing the tissue or the donor of the tissue (e.g., a brain-dead human) with a solution of the DDC in a physiologically acceptable diluent prior to excision of the tissue. Physiologically-acceptable diluents are those that are compatible with, and not harmful to, the DDC and the tissue. Such diluents are well known and include saline and other solutions and fluids.

Effective amounts of a DDC embodying features of the present invention may be determined empirically, and making such determinations lies well within the skill of the ordinary artisan. It is well understood within the art that the amount may vary as a result of one or more factors, including the type and size of the tissue, the intended use of the tissue, the length of storage of the tissue before use, the identity of any other agents being used, the number of treatments, and like factors well known in the art.

A DDC agent embodying features of the present invention may be used in conjunction with other agents to treat tissue. For example, the tissue may also be treated with preservation agents (i.e., agents which inhibit deterioration of the condition of the tissue), antibiotics, antifungal drugs, antiviral drugs, and inflammatory drugs, or other treatments (e.g., lung surfactants in the case of lung tissue). After being contacted with the DDC, the tissue may be used immediately or may be stored until needed. Methods of storing tissue are well known in the art. The tissue may be stored in contact with the DDC. Tissues are preferably stored at low temperatures, typically from 4-18° C., and non-organ tissues may typically be frozen. The time of storage will vary depending on the type of tissue, the storage environment (including the temperature of storage), and the intended use. Such times may be determined empirically, and making such determinations lies well within the skill of the ordinary artisan. Regardless of the length and conditions of storage, timely treatment with a DDC embodying features of the present invention may mitigate the effects of harvest and/or storage, and treated tissues may be in better condition than tissues not treated with a DDC.

Tissues treated with DDCs embodying features of the present invention may be used for a variety of purposes. For example, they may be transplanted into recipients. They may also be used for research purposes, such as for studying the function of the tissue.

It is understood that DDCs may induce a rest state in tissue, which lasts until the DDCs are removed. In addition, treatment of a tissue with a DDC embodying features of the present invention may improve the condition of the tissue by reducing the negative effects and consequences of harvesting and storing tissue. For example, DDCs may inhibit (prevent or reduce) the adhesion and aggregation of cells which would otherwise cause injury to a tissue (see description above). Thus, treatment of a tissue with a DDC embodying features of the present invention may prevent or reduce damage to the tissue.

In particular, ischemia (anemia due to constriction or obstruction of a blood vessel) occurs upon harvesting a tissue or an organ. Both the injury due to ischemia and that due to reperfusion after ischemia (which generally occurs upon resuming blood flow in an organ, such as when transplanting a tissue or an organ into a recipient), may be inhibited by treatment of the tissue or organ with a DDC embodying features of the present invention. To achieve maximum inhibition of ischemic injury and ischemic reperfusion injury, the tissue or organ may be contacted with the DDC before, during or after harvesting of or interruption of normal blood supply to the tissue or organ, to mitigate the rapid onset of injury and other changes associated with ischemic injury. Similarly, the tissue or organ may be contacted with the DDC before, during or after transplantation. Such treatment may have beneficial effects even for organs that are to be used immediately, such as in the case of many transplants. Preferably the contacting takes place by perfusion of the organ with a solution comprising the DDC, although rinsing and/or submersion of the tissue or organ in a medium containing the DDC may also be beneficial.

For tissues or organs that are stored (even for a short time), benefits may be obtained by contacting the tissue or organ with a DDC embodying features of the present invention immediately prior to use. This treatment may serve, among other purposes, to eliminate the effects of any cytokines that may have been produced, as well as to prevent adhesion and aggregation of cells which would otherwise cause tissue or organ injury. The treatment of a tissue or organ after storage may be the first treatment of the tissue or organ with a DDC, or may be the second treatment of the tissue or organ which was fast treated before or immediately after harvest. Again, the organ may be preferably treated by perfusion with a solution containing the DDC, although rinsing and/or submersion of the tissue or organ in a medium containing the DDC may also be beneficial.

As described above, DDCs embodying features of the present invention may suppress undesired immune responses. Thus, treatment of a tissue with a DDC prior to transplantation may act as an initial treatment for the prevention of graft rejection and/or graft versus host disease (GVHD) in transplant recipients. Of course, the recipient may receive additional amounts of a DDC in accordance with the present invention, either in single dose treatments or in multiple dose treatments, to prevent graft rejection and/or GVHD as described above. The amount administered to the recipient should also be chosen so that inhibition of injury due to ischemia and ischemia-reperfusion is continued.

Furthermore, as described above, DDCs embodying features of the present invention, administered as described above, may inhibit the transmission of pathogenic and/or viral infections from a tissue to a recipient of transplanted tissue and vice versa. This includes-all of the viral infections described above.

The invention also provides a kit for treating tissues. As used herein, the terms “kit” and “reagent kit” refer to an assembly of materials that are used in performing a method embodying features of the present invention. The reagents may be provided in packaged combination in the same or in separate containers, depending on their cross-reactivities and stabilities, and in liquid or in lyophilized form. The amounts and proportions of reagents provided in the kit may be selected so as to provide optimum results for a particular application. A reagent kit embodying features of the present invention contains at least one DDC embodying features of the present invention.

Reagents included in kits embodying features of the present invention may be supplied in all manner of containers such that the activities of the different components are substantially preserved, while the components themselves are not substantially adsorbed or altered by the materials of the container. Suitable containers include but are not limited to ampoules, bottles, test tubes, vials, flasks, syringes, bags and envelopes (e.g., foil-lined), and the like. The containers may be comprised of any suitable material including but not limited to glass, organic polymers (e.g., polycarbonate, polystyrene, polyethylene, etc.), ceramic, metal (e.g., aluminum), metal alloys (e.g., steel), cork, and the like. In addition, the containers may contain one or more sterile access ports (e.g., for access via a needle), such as may be provided by a septum. Preferred materials for septa include rubber and polymers including but not limited to, for example, polytetrafluoroethylene of the type sold under the trade name TEFLON by DuPont (Wilmington, Del.). In addition, the containers may contain two or more compartments separated by partitions or membranes that can be removed to allow mixing of the components.

Kits embodying features of the present invention may also be supplied with other items known in the art and/or which may be desirable from a commercial and user standpoint, such as instructions for treating a tissue, a container for the tissue, diluents, preservation agents, antibiotics, antifungal drugs, antiviral drugs, anti-inflammatory drugs, surfactants, buffers, empty syringes, tubing, gauze, pads, disinfectant solution, etc. Instructional materials provided with kits embodying features of the present invention may be printed (e.g., on paper) and/or supplied in an electronic-readable medium (e.g., floppy disc, CD-ROM, DVD-ROM, zip disc, videotape, audio tape, etc.). Alternatively, instructions may be provided by directing a user to an Internet web site (e.g., specified by the manufacturer or distributor of the kit) and/or via electronic mail.

It should be understood that whereas some integrin-mediated functions may be modulated by DDCs of formulae (10)-(12), (35), and (40), there may be certain integrin-mediated functions, the modulation of which is achieved by other CIMs and/or DDCs, including but not limited to one or more enantiomers of ajoene, derivatives thereof including the subgroup of (2Z)-(−)-, (2Z)-(+)-, (2E)-(−)-, and (2E)-(+)-DDCs of formula (9), compounds of formulae (2) and/or (2a), and the like. Accordingly, kits embodying features of the present invention may contain all manner of integrin modulators including trithia and/or dithia oxide structures and chiral structures, such as those described above.

The following representative procedures and Examples are provided solely by way of illustration, and are not intended to limit the scope of the appended claims or their equivalents.

### EXAMPLES

### Example 1

### Methods of Ajoene Separation into Four Enantiomers

A 3:1 mixture of racemic (Z)-(±)- and racemic (E)-(±)-ajoenes (unseparated ajoene) was dissolved in heptane/ethanol/diethylamine (90:10:0.1) and separated by HPLC on a 4.6×250 mm Chiralpak AS column (Chiral Technologies, Exton, Pa.) using a mobile phase of heptane/ethanol/diethylamine 90:10:0.1 (flow rate, 1 mL/min; temperature, 25° C.). The detector recorded two parameters: the absorption of the eluate at 254 nm and the rotation of the plane of polarization of polarized light passing through the eluate; in the latter case, upward and downward peaks corresponded, respectively, to dextrorotatory (+) and levorotatory (−) enantiomers. The resulting chromatogram contained eight, peaks superimposed in such a way that four pairs were clearly seen. In each pair, the two coinciding peaks corresponded to a particular enantiomer of ajoene—that is, (Z)-(+), (Z)-(−), (E)-(+), and (E)-(−). The (Z)-(+), (E)-(+), (E)-(−), and (Z)-(−) enantiomers of ajoene were eluted with retention times of 21.4, 23.8, 26.2, and 33.1 minutes, respectively.

In another experiment, the separation was achieved in the same system, the only difference being in the composition of the mobile phase (hexane/ethanol 90:10). Again, the (Z)-(+), (E)-(+), (E)-(−), and (Z)-(−) enantiomers were clearly separated (the respective elution times were 20.4, 23.1, 24.6, and 31.2 minutes). The peaks were collected into tared polypropylene tubes and stored on ice for 48 hours, after which analytical runs were performed with each fraction. The results of the analysis demonstrated that very good separation of the isomers had been achieved (>98%) and that the isomers did not undergo change in optical or geometric configuration under the conditions of the storage. Thereafter, the fractions were dried down by rotary evaporation (6 to 10 min at 35° C.) and re-analyzed under the same conditions. The peaks present in the fractions showed their original retention times and no other peaks were present. Thus, the drying down process did not cause change in optical or geometric configuration.

In yet another experiment, HPLC separation of the enantiomers involved a different column (10×250 mm Chiralpak AD). The conditions used were as follows: mobile phase=hexane/ethanol 90:10; flow rate=6 mL/min; temperature=ambient; detector wavelength=254 nm. Typically, 10 mg of the 3:1 mixture of ajoenes was loaded per run and fractions were collected manually. The fractions were dried down by rotary evaporation at 37° C., resulting in light oil. The oil was taken up in anhydrous ether and dried down by rotary evaporation. The resulting light oils were stored at −80° C. in sealed containers. Analytical runs on the same column demonstrated that (Z)-(+), (E)-(+), (E)-(−), and (Z)-(−) enantiomers of ajoene had retention times of 53 min, 58 min, 62 min, and 75 min, respectively.

### Example 2

### Inhibition of VLA-4-Mediated Cell Adhesion: a Method of Assessment of Integrin-Mediated Function Modulating Activity of the Four Enantiomers of Ajoene

Modulating activity of integrin-mediated functions by the enantiomers was compared in a well-defined system of inhibition of VLA-4-mediated adhesion of enzyme-labeled PMI cells (NIH AIDS Repository, Rockville, Md.) to VCAM-1-coated artificial substrata. In this experiment, enzyme-linked immunosorbent assay (ELISA) plates were coated with rabbit anti-human IgG (Fc-specific). Aliquots of 100 μL of supernatant from VCAM/IgG-secreting COST cells were added to each well, the plates incubated at 37° C. for 1 hour, and the wells washed with phosphate-buffered saline (PBS). Thereafter, 50 μL aliquots of the enantiomers (various dilutions in RPMI 1640 medium) were introduced to each well, followed by addition of 50 μL PMI cells (4×10⁶ per mL) labeled with horseradish peroxidase (HRP) by pinocytosis.

The plates were incubated at room temperature for 10 min, centrifuged (1000 rpm, 1 min) and incubated once again at 37° C. for an additional 10 min. Cells that failed to form VLA-4-dependent adhesive contacts with the substratum were washed off with PBS in two turns. Adherent cells were lyzed by adding to each well a buffered solution of the substrate, supplemented with 1% Triton X-100. The reaction was stopped with 0.5 M H₂SO₄, and the optical density of the wells was read at 450 nm. The value of this parameter, characterizing the activity of the enzyme and the PCT/US2005/007638 number of the adherent cells, is inversely proportional to the modulating activity of the compound. The (Z)-(−) enantiomer of ajoene consistently exhibited a 4-fold higher adhesion-inhibiting activity than any other enantiomer of the original 3:1 mixture of racemic Z- and racemic E-ajoenes.

### Example 3

### Differential Inhibition by Ajoene Enantiomers of Integrin-Mediated Fusion Leading to Syncytium Formation in HIV-Infected Cells

The purified enantiomers were taken up in DMSO at a concentration of 10 mg/mL. Dilutions were made in RPMI 1640 medium containing 10% fetal calf serum and 10 mM HEPES (cRPMI). H9 cells (ATCC, Rockville, Md.) infected with HIV-1_{RF} (NIH AIDS Repository, Rockville, Md.) and uninfected H9 cells were washed with cRPMI and resuspended in cRPMI at a density of 4×10⁶ per mL. Uninfected cells (50 μL) were mixed with serial dilutions of the compounds (100 μL) and incubated at 37° C. for 30 min before adding and mixing 50 μL of infected H9 cells. The plates were incubated at 37° C. for 6 to 15 hours before scoring syncytium formation.

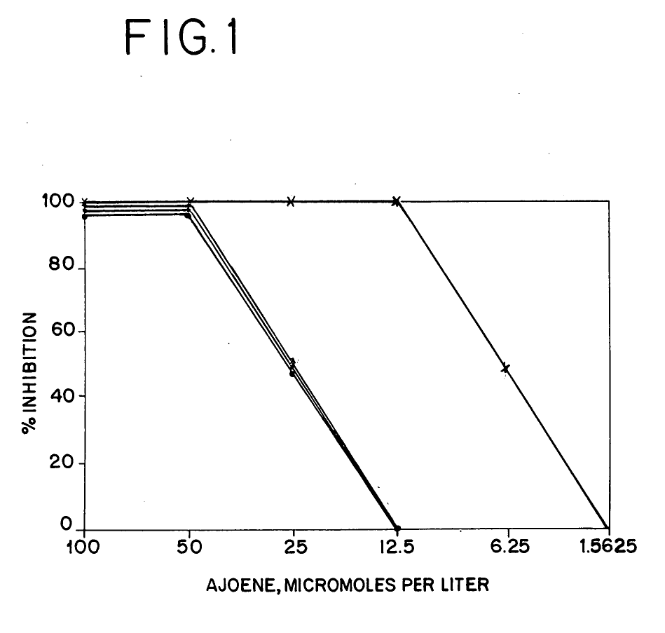

The results are presented in FIG. 1. For all the curves, the effect of ajoene enantiomers and enantiomer mixtures on the fusion of cultured, intact H9 cells with HIV-1_{RF}-infected H9 cells is disclosed. The vertical graph axis expresses the maximum amount of syncytia formed in the absence of the compounds (100 percent), while the points on the curves represent percentages of such an amount of syncytia formed in the presence of varying concentrations of the compounds and compound mixtures (micromoles per liter). The coincidence of curves 1 through 3 and the leftward shift of curve 4 demonstrate that the (Z)-(−)-enantiomer is at least four times more active than its (Z)-(+) counterpart, racemic (E)-ajoene, or the unseparated 3:1 mixture of racemic (Z)- and racemic (E)-ajoenes (the respective values of IC₁₀₀ are 12.5 and 50 micromoles per liter).

A similar result was obtained in another system, where syncytium formation was induced by mixing and incubating together for 13 hours 50 μL MT2 cells (NIH AIDS Repository) and 50 μL U937 cells (ATCC) infected with HIV-1 RT (both cell populations had the density of 2×10⁶ per mL). Taken together, Examples 2 and 3 demonstrate that (Z)-(−)-4,5,9-trithiadodeca-1,6,11-triene-9-oxide is a stereoselective, chiral integrin modulator, the activity of which is significantly higher than that of the parent racemates (E-, Z-ajoenes, and various mixtures thereof).

### Example 4

### Bioavailability of DDCS and the Active Moiety of Chiral Ajoene

As indicated in the foregoing Examples, the modulating activity of ajoene differs depending on the chiral arrangement of the sulfoxide and the geometry of the C6 double bond. Indeed, as described below, the metabolically unrelated CH₂═CH—CH₂—S— (allylthio) group of the molecule, shared by all four enantiomers, is routinely severed in biological media, with the chiral integrin-mediated function modulating moiety remaining bioavailable in relation to the activities described herein, including in the foregoing Examples.

Thus, the allylthio fragment of the ajoene molecule may be replaced, in the presence of a sulfhydryl compound (HS—R), by a suitable R with the formation of DDCs of the formula:

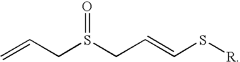

As illustrated below, an example compound of this formula is ajocysteine (a), one molecule of which is formed via the interaction of one molecule of ajoene (b) with two molecules of cysteine (c). In this reaction, the allylthio moiety of ajoene is converted to one molecule of S-allylmercaptocysteine (d) (see: “Garlic: The Science and Therapeutic Application of Allium Sativum L. and Related Species,” Lawson, L. D. et al., Eds., 1997, p. 64).

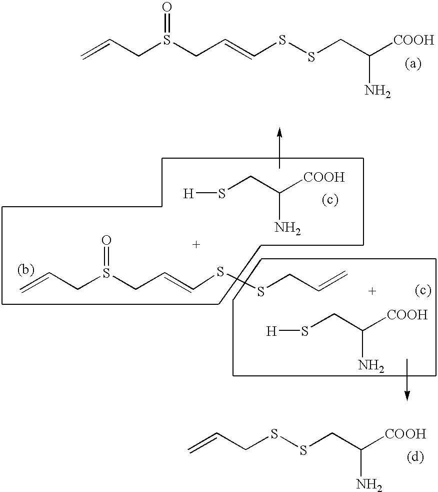

The scheme shown below illustrates one specific metabolically related setting, in which ajoene (a) is converted into DDCs when exposed to blood components; the DDC that may be formed in this reaction is (e). Allylmercaptan (c), which is derived from the allylthio fragment of ajoene (Ibidem, pp. 213-214), is unstable and undergoes enzymatic transformation to allyl methyl sulfide (d); formula (b) designates an unidentified reactant in the blood. Allyl methyl sulfide is a volatile metabolite discharged with breath (R. T. Rosen et al., “Determination of allicin, S-allylcysteine and volatile metabolites of garlic in breath, plasma or simulated gastric fluids,” J. Nutr., 2001, 131, [3S] 968S; and “Garlic: The Science and Therapeutic Application of Allium Sativum L. and Related Species” cited above, p. 66).

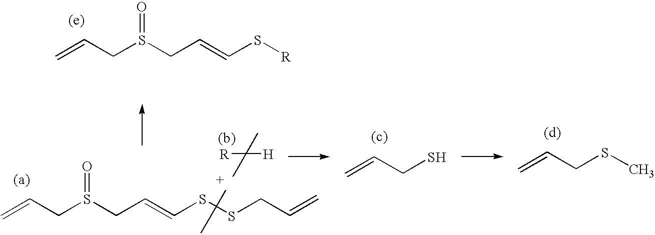

All four possible enantiomers of ajoene have a capacity to form the corresponding (2E)-(+)-, (2E)-(−)-, (2Z)-(+)-, and (2Z)-(−)-DDCs, both in vitro and in vivo.

Generally, any compound containing the core trithia oxide substructure of formula (1) is believed to be capable of forming derivatives of the following structure:

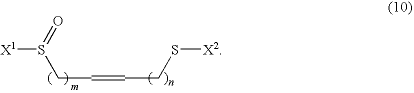

As indicated above, these compounds include all isomeric possibilities set forth herein, including enantiomeric, constituting a distinct subgroup of DDCs in accordance with the present invention.

### Example 5

### Synthesis of DDCS of General Formula (10)

A representative synthetic approach to compounds of general formula (10) in which m=0 and n=1 is illustrated in Scheme 1 below:

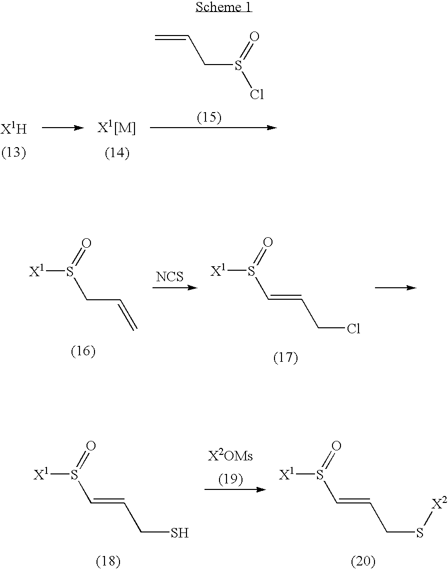

In the representative Scheme 1 shown above, a compound X¹H (13) is reacted with prop-2-ene-1-sulfinyl chloride (15) to form a sulfoxide-containing compound (16). The sulfinyl chloride (15) may be prepared from commercially available 2-propene-1-thiol, available from Aldrich (Milwaukee, Wis.) and other suppliers (e.g., Alfa Aesar, Fluka, Lancaster, Acros Organics, Pfaltz & Bauer), by oxidation followed by treatment with a chlorinating agent (e.g., thionyl chloride). Other useful starting materials analogous to sulfinyl chloride (15) include 1-alkenesulfinyl chlorides, such as those reported in the literature (e.g., Journal of Organic Chemistry 1998, 63, 7825-7832).

Depending on the structure of the X¹ group to be contained in the final product, compound (13) may either be reacted with sulfinyl chloride (15) directly or by preparing an intermediate compound (14), which may correspond to a salt of compound (13). For example, if the X¹ group of compound (13) is to be attached to the sulfoxide sulfur atom through a nucleophilic atom (e.g., oxygen, nitrogen, etc.), it may be desirable to react compound (13) with compound (15) directly, optionally in the presence of a catalyst (e.g., pyridine). Moreover, in certain instances, it may be desirable to remove an acidic proton on the X¹ group of compound (13) (e.g., by deprotonation with a base) to form a salt (14), which is then reacted with sulfinyl chloride (15). The M group in compound (14) may correspond to a wide variety of metals, including but not limited to Na, Mg, Li, Cu, and the like.

Sulfoxide-containing compound (16) is halogenated, for example with N-chlorosuccinimide (NCS), to provide chloro compound (17). Chloro compound (17) is converted to thiol (18) by reaction with, for example, excess NaSH, thiourea/water or the like. The thiol (18) is then reacted with a compound (19) or the like to introduce X² and provide the final DDC product (20), which corresponds to general formula (10) in which m=0 and n=1. Depending on the structure of the X² to be contained in the final product, the conversion of thiol (18) to DDC (20) may proceed simply using substitution-type chemistry (e.g., when the X² portion of compound (19) contains a suitable leaving group, such as mesylate, tosylate or the like).

A representative synthetic approach to compounds of general formula (10) in which m=1 and n=0 is illustrated in Scheme 2 below:

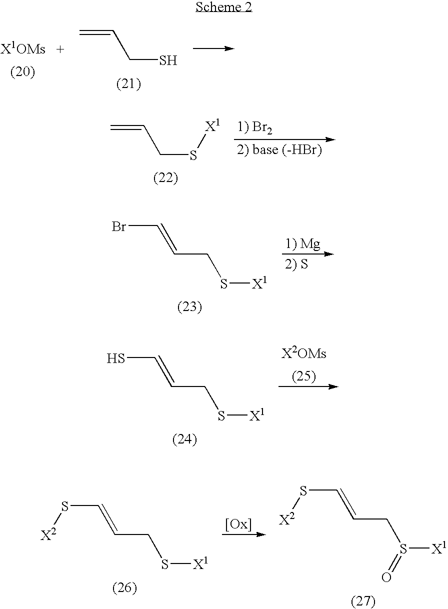

In the representative Scheme 2 shown above, a compound (20) containing a leaving group (e.g., -OMs) is reacted with commercially available 2-propene-1-thiol (21) to form compound (22). The double bond of compound (22) is brominated and then treated with base to form vinyl bromide (23) with the loss of HBr. Vinyl bromide (23) is converted to a Grignard reagent by treatment with magnesium, and the corresponding Grignard reagent is quenched with sulfur to form vinyl thiol (24). Vinyl thiol (24) is reacted with a compound such as X²OMs (25) or the like to form a disulfide compound (26). Selective oxidation of compound (26) forms final product (27), which corresponds to general formula (10) in which m=1 and n=0. The selective oxidation of one of the sulfur atoms in the presence of the other may be aided by steric accessibility of the sulfur to be oxidized. However, mixtures of oxidized products may be separated to obtain the desired oxidized product, as will be appreciated by those of ordinary skill in the art.

This above-described representative Scheme 2 may be modified in numerous ways, as will be appreciated by one of ordinary skill in the art. For example, in the synthesis of compound (27) shown above, the Grignard reagent prepared from vinyl bromide (23) may be reacted with a compound having a structure X²S—SX² to provide compound (26) directly.

An alternative representative synthetic approach to compounds of general formula (10) in which m=1 and n=0 is shown in Scheme 3 below:

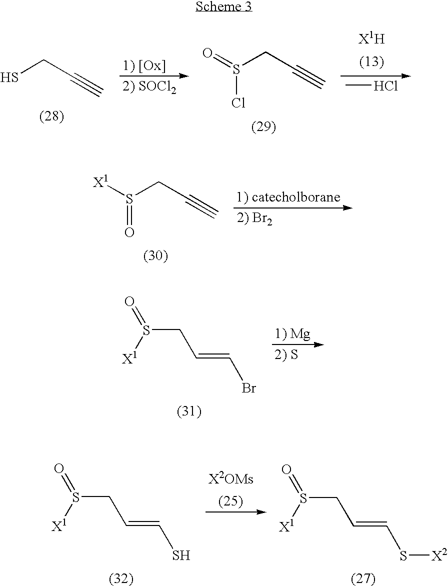

In the representative Scheme 3 shown above, propargyl thiol (28) is converted to propargyl sulfinyl chloride (29) by oxidation followed by treatment with a chlorinating agent (e.g., thionyl chloride). Propargyl thiol (28) may be prepared as reported in the literature (e.g., Synthesis 1997, 518-520). Propargyl sulfinyl chloride (29) is alkylated with a compound (13) to form sulfoxide-containing alkyne (30). Hydroboration of compound (30) (e.g., with catecholborane) and trapping with bromine gives vinyl bromide (31). Vinyl bromide (31) is converted to a Grignard reagent by treatment with magnesium, and the corresponding Grignard reagent is quenched with sulfur to form vinyl thiol (32). Dilute reaction concentrations may be employed to minimize or prevent potential undesired side reactions between Grignard moieties and sulfoxide moieties. Vinyl thiol (32) is then reacted with compound (25) to form final product (27).

The above-described representative Scheme 3 may be modified in numerous ways, as will be appreciated by one of ordinary skill in the art. For example, the sulfoxide moiety in compound 31 may be protected prior to formation of the Grignard reagent in order to prevent potential undesired reactions at the sulfoxide group.

It is to be understood that the above-described syntheses are merely representative approaches that may be modified in numerous ways, as will be appreciated by those of ordinary skill in the art. All manner of chemical transformations and reagents known in the art are contemplated for use in accordance with the presently preferred embodiments—including but not limited to those described in treatises such as Comprehensive Organic Transformations, 2^{nd} Edition by Richard C. Larock (Wiley-VCH, New York, 1999), Advanced Organic Chemistry Part B: Reactions and Synthesis by Francis A. Carey and Richard J. Sundberg (Kluwer Academic/Plenum Publishers, 2001), Some Modern Methods of Organic Synthesis, 3^{rd} Edition by W. Carruthers (Cambridge, 1987), Protective Groups in Organic Synthesis, 3^{rd} Edition by Theodora W. Greene and Peter G. M. Wuts (John Wiley & Sons, Inc., 1999), and March's Advanced Organic Chemistry, 5^{th} Edition by Michael B. Smith and Jerry March (John Wiley & Sons, Inc., 2001), and references cited therein. The entire contents of all of the above-identified treatises are incorporated herein by reference, except that in the event of any inconsistent disclosure or definition from the present application, the disclosure or definition herein shall be deemed to prevail.

In addition, the general synthetic approaches outlined above can be readily modified for use in the preparation of compounds in which the values of m and/or n are integers other than 0 and 1 by the choice of different starting materials analogous to sulfinyl chloride (15), 2-propene-1-thiol (21), and propargyl sulfinyl chloride (29).

### Example 6

### DDC Attachment to Polymeric Carriers: an Approach to Targeted Delivery

An additional group of DDCs embodying features of the present invention may be synthesized, the compounds of which share two common characteristics: (a) the polymeric nature of X² and (b) multiplicity of the active moieties attached to a single X². These compounds have a general formula:

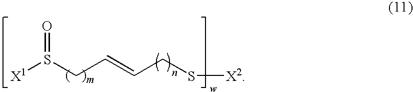

For such compounds, X² will serve as an appropriate carrier delivering the active moiety (a DDC moiety or a derivative thereof) to a designated site within an organism. In this context, appropriate carriers include but are not limited to natural or synthetic polysaccharides, proteins, polypeptides or peptides, polymers of other types not encountered in nature (e.g., polymers of fluorinated hydrocarbons), and the like. In addition, a solid phase binding group (e.g. biotin) may be used to couple a DDC functionality or a derivative thereof to a carrier (e.g., avidin, streptavidin).

For example, a compound of formula (11), wherein X² is a polysaccharide carrier, may be a polymer of furanose and/or pyranose units (optionally modified with amino or sulfhydryl groups and/or residues of acetic or sulfuric acids) carrying N-, C-, or S-linked DDC functions or their derivatives.

Furthermore, compounds of formula (11), wherein X² is a polypeptide, may include synthetic structures, the cysteine residues of which carry S-linked DDC functions or their derivatives.

The choice of a carrier creates a possibility to either facilitate the release of the active moiety or, alternatively, make the bond between it and the carrier stable. Moreover, compounds of formula (11) provide a convenient means of targeted delivery of DDC moieties. For example, a polysaccharide DDC may be conjugated with proteins (to create glycoproteins carrying DDCs), or engineered to contain specific oligosaccharide units recognized by cell surface lectins. Likewise, a polypeptide alkylated with DDC moieties may be conjugated with antibodies recognizing specific cell/tissue markers. By using this approach, DDCs may be targeted to intracellular compartments.

Compounds of formula (11) may be prepared using a variety of synthetic approaches, including but not limited to strategies analogous to those outlined in Schemes 1-3 above. One representative synthetic approach to compounds of formula (11) is shown in Scheme 4 below:

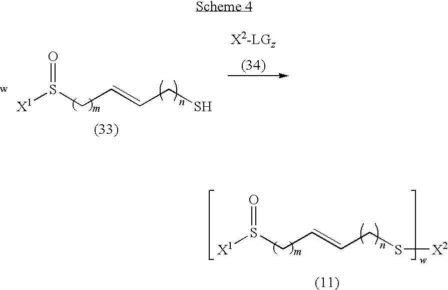

In the representative Scheme 4 shown above, a plurality (w) of DDC thiols (33) are reacted with a carrier (34) that contains a plurality (z) of leaving groups (LG). Preferably, the carrier (34) contains at least as many leaving groups as the number of DDC fragments to be attached thereto (i.e., z≧w). In this representative Scheme 4, attachment of DDC fragments to the carrier is achieved by substitution-type chemistry analogous to the preparations of compounds (20), (26), and (27) shown above in Schemes 1, 2, and 3, respectively. It should be noted that the individual DDC thiols (33) to be attached to the carrier (34) need not be identical and that the plurality of compounds (33) may include compounds containing X¹ groups that are the same or different. Similarly, the m and/or n values of the plurality of compounds (33) may be the same or different. Furthermore, the individual DDC thiols (33) may include mixtures of stereoisomers or individual isomers, such as the (E,R), (E,S), (Z,R), and (Z,S) isomers.

### Example 7

### Polymeric DDCs

An additional group of DDCs embodying features of the present invention may be synthesized in which a plurality of DDC moieties, which may be the same or different, are incorporated into a polymeric structure. These compounds have a general formula:

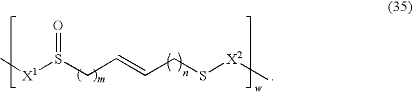

The variables m, n, X¹, and X² may be as defined above for the DDCs embodying features of the present invention. The groups X¹ and X² may be the same or different and include at least one functional group, preferably at a terminal position, which is capable of participating in a polymerization-type reaction (e.g., chain-growth, step-growth, etc.) either with a functional group within the same monomeric unit or with a functional group in a different monomeric unit. The variable w is an integer from 2 to 1000.

One representative synthetic approach to compounds of general formula (35) is shown in Scheme 5 below:

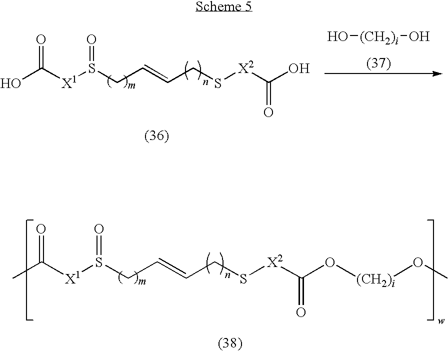

In the representative Scheme 5 shown above, a DDC compound (36) is reacted with an α,ω-diol (37) to form a polymeric structure (38). Each of the X¹ and X² portions of compound (36) contains a carboxylic acid, preferably a terminally located one, which may react with one of the hydroxyl functions of the α,ω-diol (37), thereby forming an ester linkage. The distance between DDC moieties in the polymeric backbone may be controlled by varying the length of the alkylene portion of the α,ω-diol (37). Preferably, the variable i in α,ω-diol (37) is between 1 and 30. In addition, it is presently preferred that the X¹ and X² moieties be sufficiently long to minimize or prevent any adverse interaction with the adjacent carbonyl groups that could otherwise reduce the efficacy of the DDC units in the polymeric chain. In addition, it is presently preferred that the X¹ portion of compound (36) is alkenyl and that at least one double bond in the alkenyl X¹ fragment is allylic to the sulfoxide group in order to resemble the structure of the terminal double bond allylic to the sulfoxide group in ajoene. Thus, a presently preferred structure for compound (36) has a formula:

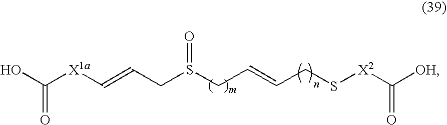

wherein X^{1a} may be as defined above for the X¹ portion of DDCs embodying features of the present invention.

It should be noted that while the polymerization chemistry illustrated in representative Scheme 5 above is based on a step-growth polymerization with formation of polyester linkages, all manner of polymerizations known in the art (e.g., formation of polyamides, polycarbonates, polyurethanes, epoxy resins, polymerizations of alkenes and alkynes, free radical polymerizations, anionic polymerizations, cationic polymerizations, and the like) may be adapted to and are contemplated for use in accordance with the present invention. Representative polymerizations useful in accordance with the present invention include but are not limited to those described in the treatise entitled Principles of Polymerization, 4^{th} Ed. By George Odian (John Wiley & Sons, Inc., New York, 2004), the entire contents of which are incorporated herein by reference, except that in the event of any inconsistent disclosure or definition from the present application, the disclosure or definition herein shall be deemed to prevail.

An additional group of polymeric DDCs embodying features of the present invention has a general formula:

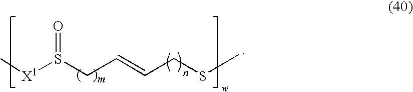

The plurality (w) of DDC moieties in compound (40) may be the same or different. The variables m, n, and X¹, and w may be as defined above. In the compounds of formula (40), the sulfur atom of one DDC moiety is directly attached to the X¹ group of another DDC moiety. The group X¹ includes at least one functional group, preferably at a terminal position, which is capable of participating in a polymerization-type reaction (e.g., chain-growth, step-growth, etc.) with a sulfur atom.

One representative synthetic approach to compounds of general formula (40) is shown in Scheme 6 below:

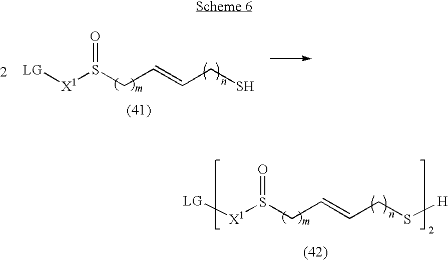

As shown in the representative Scheme 6 above, a dimeric DDC (42) may be prepared by the intermolecular coupling of two molecules of thiol (41). The X¹ portion of thiol (41) contains a suitable leaving group LG (e.g., mesylate, tosylate or the like), which may react with the thiol end of a second molecule, or the salt thereof, by substitution-type chemistry. The dimeric DDC (42) may then be further reacted with additional thiol (41) or a salt thereof to introduce a third DDC subunit, thereby increasing the length of the oligomeric chain. Undesired intramolecular cyclization of dimer (42) or higher oligomers may be minimized or prevented by the reaction conditions used (e.g., concentrations of reactants). In addition, the geometry of the central double bond in thiol (41) and/or the structure of the X¹ moiety may likewise decrease the possibility of competitive intramolecular cyclizations (e.g., an E configuration for the central double bond of thiol (41) and dimer (42) will hold the reactive centers apart, thereby reducing the tendency towards intramolecular cyclization).

It should be noted that while the coupling chemistry illustrated in representative Scheme 6 above is based on a step-wise increase of the oligomeric chain using substitution-type couplings, additional methodologies, such as the addition of thiols to alkenes or alkynes (e.g., by electrophilic, nucleophilic or free-radical mechanisms), may also be employed and are likewise contemplated for use in accordance with the present invention. In these synthetic approaches, the X¹ portion of thiol (41) may contain a double or triple bond instead of leaving group LG.

### Example 8

### DDC Attachment to Particulate Material

In certain cases, targeted delivery is most efficient when particulate material (e.g., microspheres, beads, liposomes, micelles, etc.) to which the active agents are attached is used as a carrier. Polymeric DDCs of formula (11) may be readily immobilized on surfaces of beads, using techniques developed for (poly)peptides, (poly)saccharides, and (glyco)proteins. Obtaining stable preparations of liposomes carrying DDCs may involve their conversion into lipids capable of forming liposomes (i.e., phospholipids, sphingolipids, glycolipids, or sterols).

For example, if X¹ in formula 10 is allyl, X² should be selected from substituted phosphoglyceryls, furanose/pyranose units, and polycyclic aryls, in order to obtain, respectively, a phospholipid/sphingolipid, a glycolipid, or a sterol modified with a DDC function (e.g., via S-alkylation, N-alkylation, O-alkylation, or O-acylation). Incorporation of such modified lipids into liposomes or micelles gives a particulate form of DDCs having the formula:

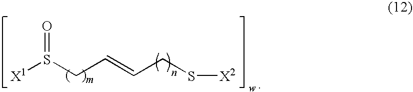

### Example 9

### Dimeric DDCs Linked Through a Disulfide Bond

An additional group of DDCs embodying features of the present invention may be synthesized in which two DDC moieties, which may be the same or different, are incorporated into a dimeric structure linked through a disulfide bond. These compounds have a general formula (43):

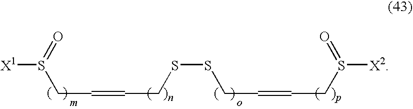

The variables X¹ and X² may be the same or different and may be as defined above for the DDCs embodying features of the present invention. The variables m, n, o, and p may each be independently selected from the group of integers from 0 to 30. In a presently preferred embodiment, X¹=allyl, m=1, n=0, X²=allyl, p=1, and o=0, such that the compound (43) represents a dimeric structure containing two DODOyl fragments corresponding to the active moieties of ajoene.

It is to be understood that compounds of general formula (43) include all of the possible optical and geometric isomers. Accordingly, cleavage of the disulfide bond of compound (43) may result in the formation of several different active moieties having different chiral and/or geometric configurations, depending on the chiral and/or geometric purity of the starting compound. As used herein, isomers of compound (43) are identified sequentially from left to right by (1) the configuration of the central sulfoxide in the left half of the molecule, (2) the configuration of the central double bond in the left half of the molecule, (3) the configuration of the central double bond in the right half of the molecule, and (4) the configuration of the central sulfoxide in the right half of the molecule. For example, a specific isomer of compound (43) will be designated as (R or S, E or Z, E or Z, R or S).

Compounds of general formula (43) may be readily prepared from the corresponding monomeric thiols by oxidation (e.g., with H₂O₂, CuSO₄, Me₂SO—I₂ or the like) or by alternative methodologies including but not limited to the reaction of the corresponding monomeric alkyl halides with disulfide anions.

DDCs of general formula (43) may have particular therapeutic utility inasmuch as one molecule of general formula (43) provides a “double dose” of DDC moieties. The individual DDC units may be readily liberated by cleavage of the disulfide bond.

### Example 10

### Dimeric DDCS Linked Through an Intermediary Moiety

An additional group of DDCs embodying features of the present invention, which are related to compounds of formula (43) above, may be synthesized in which two DDC moieties, which may be the same or different, are incorporated into a dimeric structure linked through an intermediary moiety X³. These compounds have a general formula (44):

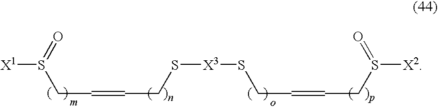

The variables X¹ and X² may be the same or different and may be as defined above for the DDCs embodying features of the present invention. The variables m, n, o, and p may each be independently selected from the group of integers from 0 to 30. The variable X³ is a linking moiety capable of forming bonds (e.g., covalent and/or ionic) with sulfur atoms of the two DDC moieties. By way of example, X³ may be a divalent moiety such as an alkylene group, for example —(CH₂)_{q}—, where q is an integer from 1 to 30. Such compounds may be readily prepared from the corresponding thiols by substitution-type chemistry, as described above. Alternatively, X³ may be a positively charged cationic species, such as a metal atom, preferably with a charge of at least +2, to counterbalance the negative charges of the two thiolate anions (i.e., S⁻) of the DDC portions of the molecule. Such compounds may be prepared by deprotonating the thiol hydrogens in the corresponding thiols to form thiolate anions, and by forming a salt with a suitable cation (e.g., Zn²⁺, Fe²⁺, Cu²⁺, Co²⁺, etc.). Of course, cationic species having charges other than +2 may also be used, including but not limited to cations with charges of +3 and +4. With such higher charge cations, three or more DDC units may be associated with the salts of general formula (44).

In a presently preferred embodiment, X¹=allyl, m=1, n=0, X²=allyl, p=1, and o=0, such that the compound (44) represents a dimeric structure containing two DODOyl fragments corresponding to the active moieties of ajoene, and X³=—(CH₂)_{q}—, where q is selected from the group consisting of 1, 2, 3, 4, 5, and 6.

The foregoing detailed description and representative examples have been provided solely by way of explanation and illustration, and are not intended to limit the scope of the appended claims. Many variations in the presently preferred embodiments illustrated herein will be obvious to one of ordinary skill in the art, and remain within the scope of the appended claims and their equivalents.

1-108. (canceled)

109. An integrin-modulator comprising:
a compound that forms at least one moiety in vivo, wherein the moiety has a structure: 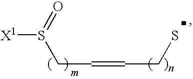
wherein:
X¹ is selected from the group consisting of hydrogen, an alkyl, an alkenyl, an alkynyl, an aryl, an aryl substituted with one or more —NO₂ groups, an aryl substituted with one or more lower alkyls, a group of formula RO—, a group of formula RC(O)—, a group of formula RC(O)O—, a group of formula ROC(O)—, a group of formula (R)₂N—, a group of formula RC(O)N—, a group of formula R(NH)C(O)—, a group of formula RN═N—, a group of formula RS—, a group of formula RSO₂—, a group of formula RS(O)—, RSC(O)—, a group of formula RSO2O—, a group of formula RS(O)O—, a halogen atom, a nitroso group, a furanose unit, a pyranose unit, a combination of furanose units, a combination of pyranose units, a combination of furanose and pyranose units, and combinations thereof;
R is selected from the group consisting of a hydrogen, a lower alkyl, a lower alkenyl, an aryl, an aryl substituted with one or more lower alkyls, S-cysteinyl, peptidyl, an alkylphosphoglyceryl, an alkenylphosphoglyceryl, or an acylphosphoglyceryl;
m is an integer from 0 to 30; and
n is an integer from 0 to 30;
providing that the compound is not ajoene or ajocysteine.

110. The invention of claim 109, wherein X¹ is alkenyl.

111. The invention of claim 109, wherein X¹ is CH₂═CH—CH₂—.

112. The invention of claim 109, wherein the radical is a (E,R) stereoisomer.

113. The invention of claim 109, wherein the radical is a (E,S) stereoisomer.

114. The invention of claim 109, wherein the radical is a (Z,R) stereoisomer.

115. The invention of claim 109, wherein the radical is a (Z,S) stereoisomer.

116. The invention of claim 109, wherein the compound comprises at least two stereoisomers having a configuration selected from the group consisting of (E,R), (E,S), (Z,R), (Z,S), and combinations thereof.

117-173. (canceled)
